(19) United States
(12) Patent Application Publication
(10) Pub. No.: US 2026/0171204 A1
(43) Pub. Date: Jun. 18, 2026

(54) SKIN BIOMARKER DIAGNOSES AND TREATMENTS

(71) Applicant: L'Oreal, Paris (FR)

(72) Inventors: Nicholas Sinclair, Clark, NJ (US); Fred Orsita, Wayne, NJ (US)

(73) Assignee: L'Oreal, Paris (FR)

(21) Appl. No.: 19/420,527

(22) Filed: Dec. 15, 2025

Related U.S. Application Data
(60) Provisional application No. 63/734,576, filed on Dec. 16, 2024.

(30) Foreign Application Priority Data
Feb. 14, 2025 (FR) 2501560

(51) Int. Cl. G16H 20/00

(52) U.S. Cl. CPC G16H 20/00

(57) ABSTRACT

Systems, devices, and methods for skin biomarker analysis and computation of predicted responsiveness to skincare treatments for management of skin health. A system is configured to receive a measurement of one or more skin biomarkers, compute a predicted beauty outcome that is predicted to occur due to a course of action, and recommend or not recommend the course of action, based on the responsiveness metric, such that the skin health of the individual is advanced toward a desired beauty outcome. Systems can recommend products, services, and other courses of action such as lifestyle changes, to improve the health of the individual.

### SUMMARY

In an aspect, the disclosure provides a system configured for management of skin health of an individual, the system comprising: circuitry configured to receive a measurement of one or more skin biomarkers of the individual and compute, based on the measurement, a predicted beauty outcome that is predicted to occur due to a course of action; circuitry configured to receive a desired beauty outcome for the individual and compute, based on the predicted beauty outcome and the desired beauty outcome, whether the course of action would advance the skin health of the individual toward the desired beauty outcome for a responsiveness metric; and circuitry configured to recommend or not recommend the course of action to the individual, based on the responsiveness metric, such that the skin health of the individual is advanced toward the desired beauty outcome.

In embodiments, the measurement of the one or more skin biomarkers of the individual is transmitted from a measurement device and received by a computational device.

In embodiments, the measurement device comprises an on-site biomarker reader configured for on-site measurement of the one or more skin biomarkers of the individual.

In embodiments, the computational device comprises a networked server, a cloud computing system, a smartphone, a tablet, a desktop computer, a laptop computer, a smart watch, a wearable device, or any combination thereof.

In embodiments, a first threshold of the measurement corresponds with a positive responsiveness metric and predicted responsiveness of skin of the individual to the course of action, and wherein a second threshold of the measurement corresponds with a negative responsiveness metric and predicted non-responsiveness of skin of the individual to the course of action.

In embodiments, the course of action comprises: a lifestyle change, a cessation of a negative habit, a reinforcement of a positive habit, a selection of a product, a selection of a service, an application of a product to skin of the individual, or any combination thereof.

In embodiments, the desired beauty outcome is computed as an assumed desired beauty outcome for the individual based on one or more characteristics of the individual, as an expressed desired beauty outcome based on one or more inputs from the individual, or any combination thereof.

In embodiments, the system comprises circuitry configured to receive an imagery measurement of skin of the individual, an electromechanical measurement of skin of the individual, or both, and circuitry configured to compute the responsiveness metric based on the imagery measurement, the electromechanical measurement, or both.

In embodiments, the imagery measurement is transmitted from an image capture device and received by a computational device, and wherein the electromechanical measurement is transmitted from an electromechanical device and received by the computational device.

In embodiments, the system comprises circuitry configured to receive a plurality of measurements of a plurality of skin biomarkers of the individual and compute, based on the plurality of measurements, the predicted beauty outcome that is predicted to occur due to the course of action.

In embodiments, the circuitry computes the predicted beauty outcome based on measurements of one or more relatively up-regulated skin biomarkers, measurements of one or more relatively down-regulated skin biomarkers, or any combination thereof.

In embodiments, the responsiveness metric is computed based at least in part on: a skin health characteristic of skin of the individual selected from the group consisting of: a skin texture, a cornification level, a presence or absence of an eye wrinkle, a level of insulin metabolism, a level of skin evenness or elastosis, a degree of loss of elasticity of skin, an antimicrobial property, a presence or absence of a dilated pore, a degree of inflammation, and any combination thereof; a predicted response of skin of the individual to a retinol treatment selected from the group consisting of: retinol (vitamin A₁), C-beta-D-xylopyranoside-2-hydroxy-propane (C-Xyloside or ProXylane®), azelaic acid, and any combination thereof; a longevity characteristic of the individual selected from the group consisting of: a degree of inflammation, a level of stem cell exhaustion, a degree of DNA instability, a degree of proteostasis, a degree of epigenetic change, a level of microbiome change, a level of mitochondrial function, a level of cellular communication, a level of cellular senescence, and any combination thereof.

In embodiments, the responsiveness metric is computed based at least in part on a skin health characteristic of skin of the individual and the one or more skin biomarkers comprises a protein selected from the group consisting of: filaggrin 2 (FLG2), Insulin-degrading enzyme (IDE), epidermal transglutaminase (TG3), lipocalin-1 (LCN1), Chitinase-3-like protein 1 (YKL40), and any combination thereof.

In embodiments, the responsiveness metric is computed based at least in part on a predicted response of skin of the individual to a retinol treatment and the one or more skin biomarkers comprises a protein selected from the group consisting of: Retinoic acid receptor responder protein 1 (RARRES1), Aldehyde dehydrogenase family 3 member A2 (ALDH3A2), N-ribosyldihydronicotinamide:quinone dehydrogenase 2 (NQO2), and any combination thereof.

In embodiments, the responsiveness metric is computed based at least in part on a longevity characteristic of the individual and the one or more skin biomarkers comprises a protein selected from the group consisting of: lamin A/C (LMNA), galactosidase beta 1 (GLB1), serpin family A member 1 (SERPINA1), lamin B1 (LMNB1), peptidase inhibitor 3 (PI3), catalase (CAT), superoxide dismutase 1 (SOD1), cadherin 1 (CDH1), CD44 molecule (IN blood group) (CD44), ras homolog family member A (RHOA), cell division cycle 42 (CDC42), transporter 1, ATP binding cassette subfamily B member (TAP1), filaggrin (FLG), keratin 1 (KRT1), one or more keratin genes (KRTs), keratin 14 (KRT14), keratin 15 (KRT15), keratin 16 (KRT16), keratin 17 (KRT17), interleukin-1 alpha (IL-1α), interleukin-18 (IL-18), Kallikrein Related Peptidase 5 (KLK5), Kallikrein Related Peptidase 7 (KLK7), and any combination thereof.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of the claimed subject matter will become more readily appreciated as the same become better understood by reference to the following detailed description, when taken in conjunction with the accompanying drawings, wherein:

FIG. 1 shows examples of inputs, analytes, algorithms, skin health characteristics, outputs, and courses of action of a system configured for management of skin health of an individual, according to aspects of the disclosure;

FIG. 2 shows further examples of inputs, analytes, algorithms, skin health characteristics, outputs, and courses of action of a system configured for management of skin health of an individual, according to aspects of the disclosure;

FIG. 3 shows examples of steps for use of a system for management of skin health, including with generation of a recommended course of action, according to aspects of the disclosure;

FIG. 4 shows examples of skin biomarkers and skin health characteristics thereof, retinol response skin biomarkers, and longevity skin biomarkers, according to aspects of the disclosure;

FIG. 5A shows results from a descriptive analysis of clinical signs in an example population, according to aspects of the disclosure. The figure shows hierarchical clustering of the (N=21) assayed clinical signs in the full example population. The number of clusters to retain was calibrated using the Jaccard's index as a measure of cluster similarity. The optimal number of clusters was defined as the one maximizing the decrease in within-cluster Jaccard's index;

FIG. 5B shows results from a descriptive analysis of clinical signs in an example population, according to aspects of the disclosure. The figure shows results from a series of univariate logistic regressions for the risk of each of the (N=21) clinical sign as the outcome. For each clinical sign we report the Odds Ratios (OR) and 95% confidence intervals, adjusting for center;

FIG. 6A shows results from protein profiling, according to aspects of the disclosure. The figure shows a heatmap of pairwise Pearson's correlation coefficients for each of the (N=5) proteins;

FIG. 6B shows results from protein profiling, according to aspects of the disclosure. The figure shows a network representation of a series on LASSO-penalized logistic models regressing the concentrations of the five measured proteins against the risk of each (N=21) clinical signs, separately. Results are presented for models adjusted for center. Proteins are represented as nodes and clinical signs are indicated according to the (N=3) clusters of co-occurring clinical signs, including wrinkles, oily nature of the skin, and skin dryness. Proteins that are connected to a given clinical sign are those that were stably selected as jointly and complementarily explanatory of the risk of that clinical sign;

FIG. 6C shows results from protein profiling, according to aspects of the disclosure. The figure shows a network representation of a series on LASSO-penalized logistic models regressing the concentrations of the five measured proteins against the risk of each (N=21) clinical signs, separately. Results are presented for models adjusted for chronological age. Proteins are represented as nodes and clinical signs are indicated according to the (N=3) clusters of co-occurring clinical signs, including wrinkles, oily nature of the skin, and skin dryness. Proteins that are connected to a given clinical sign are those that were stably selected as jointly and complementarily explanatory of the risk of that clinical sign;

FIG. 7 shows results showing association between chronological age and protein concentrations, according to aspects of the disclosure. Regression coefficients (95% confidence intervals) and p-values were calculated from a series of linear regressions with age as predictor and standardized protein concentrations as outcome. Significant associations after correction for multiple testing using a Bonferroni corrected per test significance level ensuring a family wise error rate below 0.05 are represented in bold;

FIG. 8A shows clinical age and its determinants, according to aspects of the disclosure, showing clinical age is defined from a stability selection LASSO using the (N=21) clinical signs as predictors and chronological age as (continuous) outcome. The per-clinical selection proportion as defined by the number times that features were included in the model across (N=1000) sub-samples of the data (top panel) is reported. The threshold in selection proportion to define stably selected predictors was calibrated jointly with the penalty parameter and is represented as a horizontal dotted line. The clinical signs with selection proportion above that threshold are considered those reported as stably selected and their label in presented on the X-axis. The effect size estimated from a recalibrated model fitting a linear regression with stably selected clinical signs as predictors (bottom panel) is also reported;

FIG. 8B shows clinical age and its determinants, according to aspects of the disclosure, showing Receiver Operating Characteristic (ROC) curves for the recalibrated logistic models fitted on (N=1000) independent 25% testing sets including stably selected proteins. The results for the model are shown including center and levels of (i) IDE, (ii) LCN1, and (iii) both IDE and LCN1;

FIG. 8C shows clinical age and its determinants, according to aspects of the disclosure, showing median, 5th and 95th percentiles of the AUC from logistic models for dichotomized age acceleration indicator in models sequentially including each protein in decreasing order of their selection proportion;

FIG. 9 shows an association between age A/D and protein concentrations, according to aspects of the disclosure. Regression coefficients (95% confidence intervals) and p-values are calculated from a series of linear regressions with each of the protein concentrations as predictor and age A/D (clinical age-chronological age) as outcome. Significant associations after correction for multiple testing using a Bonferroni corrected per test significance level ensuring a family wise error rate below 0.05 are represented in bold. Models are all adjusted for center;

FIG. 10A shows a perspective view of an example biomolecule extraction device, according to aspects of the disclosure;

FIG. 10B shows an exploded perspective view of the biomolecule extraction device, according to aspects of the disclosure;

FIG. 10C shows a perspective view of an example biomolecule extraction device, according to aspects of the disclosure;

FIG. 10D shows a process diagram illustrating an example process of extracting protein using the biomolecule extraction device, according to aspects of the disclosure;

FIG. 11 shows a diagram of an example computational device, according to aspects of the disclosure; and

FIG. 12 shows a flow chart of an example process for generation of a recommendation with use of a system configured for management of skin health of an individual, according to aspects of the disclosure.

| Drawings | |
|---|---|
| Label | Description |
| 10 | Tape for obtaining samples |
| 100 | Insertion part |
| 110 | First fixing part |
| 120 | Second fixing part |
| 130 | Sealing part |
| 132 | Reinforcing ribs |
| 140 | Detachable sealing cap |
| 200 | Body part |
| 210 | Chamber |
| 212 | Pressing part |
| 218 | Shielding film |
| 220 | Base |
| 230 | Introduction part |
| 320 | Expanded discharge port |
| 400 | Insertion part |
| 410 | Fixing part |
| 430 | Sealing part |
| 500 | Body part |
| 510 | Chamber |
| 512 | Pressing part |
| 530 | Introduction part |
| 550 | Connection part |
| 600 | Discharge port |
| 700 | Skin biomarker extraction process |
| 701 | Individual's face contacted with tape for obtaining |
| | samples |
| 702 | Tape with sample placed between first and second |
| | fixing parts |
| 703 | Tape with sample processed with solution for sample |
| | extraction |
| 704 | Sample extracted |
| 705 | Detachable sealing cap removed |
| 706 | Device inverted and sample passed through expanded |
| | discharge port and into measurement device or |
| | cartridge thereof |
| 1000 | Biomolecule extraction device |
| 2000 | Biomolecule extraction device |
| 3500 | Computing device diagram |
| 3502 | Processor(s) |
| 3504 | Storage medium(s) |
| 3506 | Network interface(s) |
| 3508 | Communication bus(es) |
| 3510 | System memory(s) |
| 4000 | Process for generation of a recommendation with use |
| | of a system configured for management of skin health |
| | of an individual |
| 4100 | Measure levels of one or more skin biomarkers |
| 4200 | Compute a correlation between a biomarker |
| | measurement and a predicted beauty outcome due to |
| | a course of action |
| 4300 | Is predicted beauty outcome consistent with desired |
| | beauty outcome (YES or NO)? |
| 4350 | YES |
| 4400 | NO |
| 4500 | Generate a recommendation that includes the course |
| | of action |
| 4550 | Generate a recommendation that does not include the |
| | course of action |

### DETAILED DESCRIPTION

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed.

Skincare consumers, cosmetic professionals, and other individuals are desirous of skincare products, services, and routines that are consistently effective. The effectiveness of a skin health treatment balances on the treatment itself as well as the ability of the skin to benefit from the treatment. A range of genetic, genomic, epigenetic, epigenomic, innate, and environmental variables, which can be relevant for a particular individual, contribute to the ability of the skin of the individual to benefit from a given treatment. Prior efforts may have resulted in cosmetic product recommendations being given to at least some individuals whose biological background may have contributed to a less-than-effective response to a given treatment with the cosmetic product. As a result, those individuals may have experienced dissatisfaction with the product or treatment and, possibly, may have discontinued efforts to experiment with or improve their skin health, appearance, or perceived beauty. For many individuals, evidence-based skincare recommendations would help them have the confidence to try new skincare treatments or maintain existing routines. Accordingly, there is a need for high-value, innovative approaches that leverage non-intrusive personal biological information, such as skin biomarker measurements, for prediction of the effectiveness of a given skincare treatment for improved skincare guidance, product or treatment suggestions, and the like. The present disclosure addresses these and other long-felt and unmet needs in the art.

The disclosure provides systems, devices, and methods that implement novel biomarker measurements for the diagnosis of skin conditions, evaluation of skin statuses, and prediction of responsiveness of the skin of an individual to one or more skincare treatments. The biomarker approaches can be combined with other skincare information, such as skin or facial imagery, biomechanical measurement of the skin, hydration or electrical resistivity of the skin, measurement of oils, or the like, for combinatorial methods of skin diagnosis and treatment. The disclosure also provides for the validation of skin biomarker measurements as inputs for determination of skincare products, treatments, routines, and the like, that are predicted or expected to be effective for a particular individual, or group of individuals, with particular skin biomarker profiles or measurements.

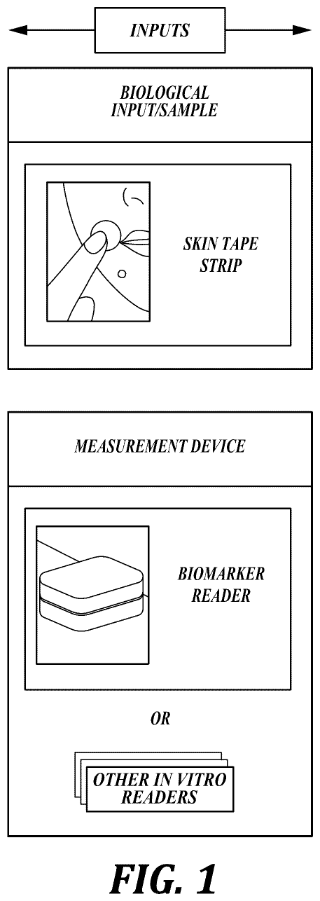

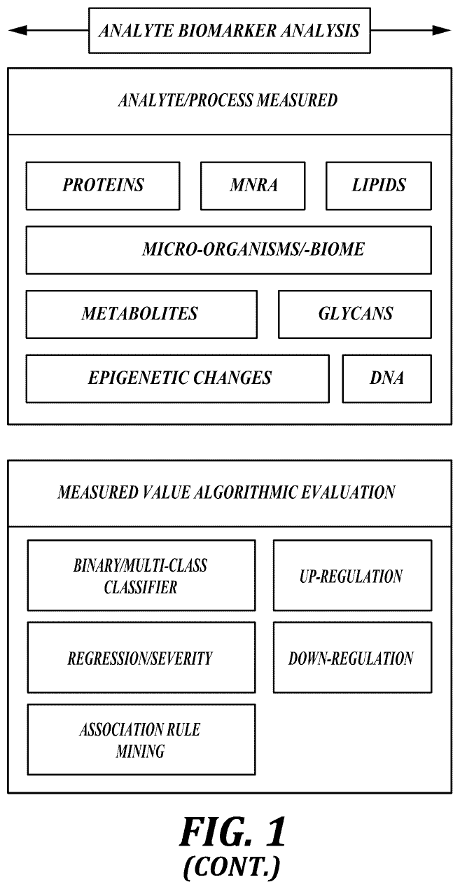

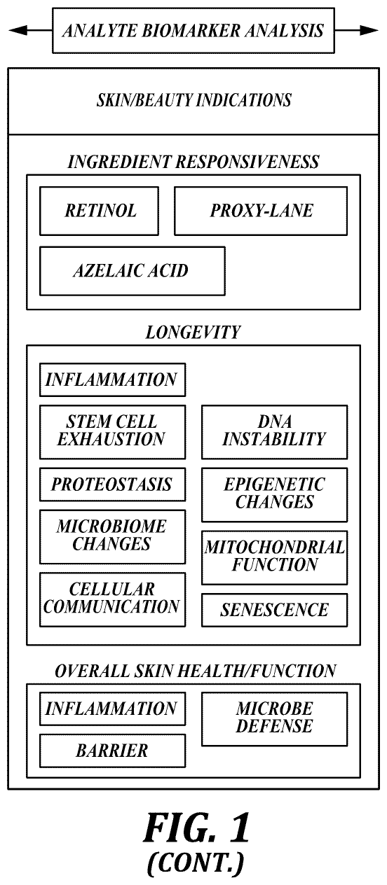

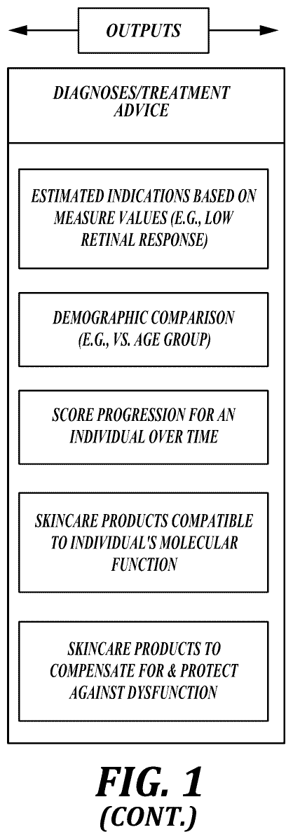

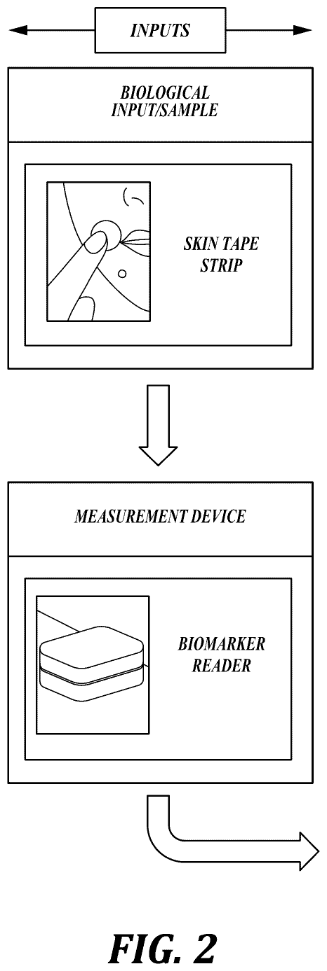

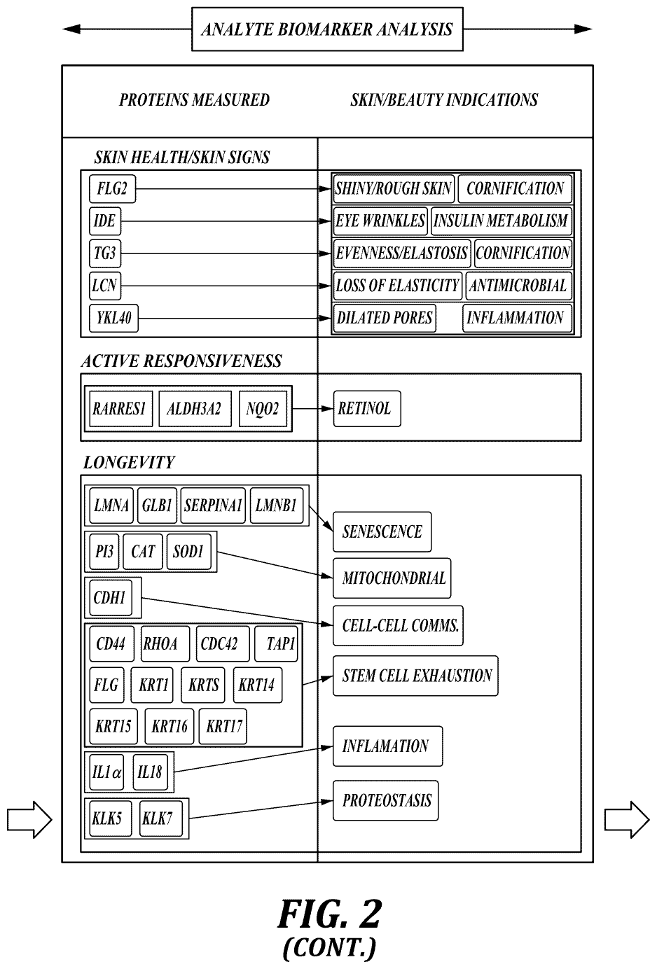

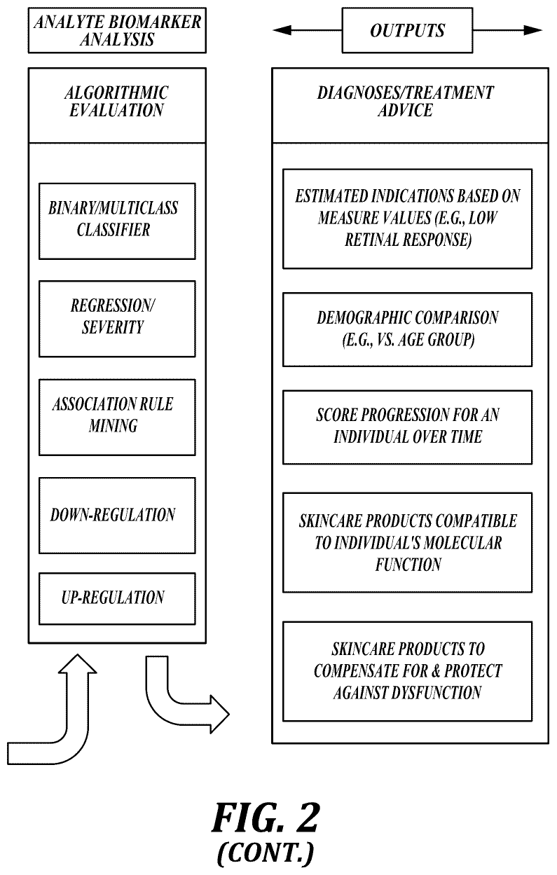

In a general aspect, a system of the disclosure accepts as an input one or more skin biomarker measurements, and computes as an output one or recommendations for a skincare treatment or cosmetic therapy. As shown at FIGS. 1 and 2, there are various example inputs, analytes, algorithms, skin health characteristics, outputs, and courses of action of a system configured for management of skin health as provided by the disclosure. A system is generally configured for management of skin health of an individual based at least in part on skin biomarker measurement data. The system comprises circuitry configured to receive a measurement of one or more skin biomarkers of the individual (e.g., as produced by a measurement device or biomarker reader analysis of a skin tape strip containing a skin sample). Non-limiting examples of measurement devices that can be used with a system of the disclosure include those as described in U.S. Pat. No. 10,520,404 B2, which is incorporated by reference herein in its entirety for all purposes. Circuitry of the system can also compute, based on the measurement, a predicted beauty outcome that is predicted by the system to occur due to a course of action. The system also comprises circuitry configured to receive a desired beauty outcome for the individual and compute, based on the predicted beauty outcome and the desired beauty outcome, whether the course of action would advance the skin health of the individual toward the desired beauty outcome for a responsiveness metric. The system further comprises circuitry configured to recommend, or not recommend, the course of action to the individual, based on the responsiveness metric. In this manner, the skin health of the individual is advanced toward the desired beauty outcome (e.g., whether or not the individual takes the course of action).

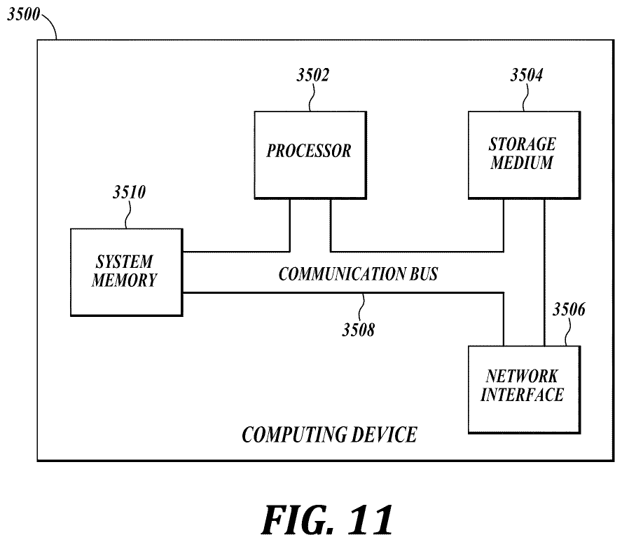

In various embodiments, circuitry of the system can be configured as one or more computational devices of the system, for example, as one or more smartphones, mobile devices, tablets, smart watches, laptop computers, desktop computers, networked servers, another computing device, or any combination thereof. These devices and their equivalents can be configured to perform these and other steps of methods of the disclosure with execution of software by one or more processors. In various embodiments, the software can be stored as processor-executable instructions on a non-transitory computer-readable medium, such as a memory, of the one or more computational devices. For example, FIG. 11 shows a diagram of a computational device that can comprise circuitry for carrying out all or part of methods of the disclosure. An example computing device 3500 can comprise, as circuitry, a processor 3502, a system memory 3510, a storage medium 3504, and a network interface 3506, interconnected by a communication bus 3508. Other configurations of computing devices are contemplated without departing from the scope and spirit of the disclosure.

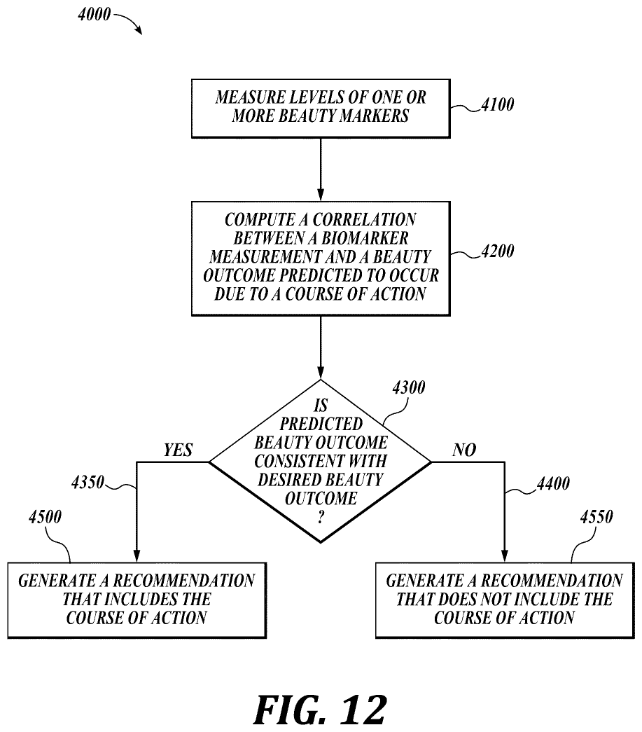

As shown at the flow chart of FIG. 12, an example process for generation of a recommendation with use of a system configured for management of skin health of an individual involves the use of biomarker measurement data. A process 4000 comprises, at step 4100, measuring levels of one or more beauty biomarkers; at step 4200, computing a correlation between a biomarker measurement and a beauty outcome predicted to occur due to a course of action; and at step 4300, computing whether the predicted beauty outcome is consistent with the desired beauty outcome. If the predicted beauty outcome is consistent with the desired beauty outcome 4350 (step 4300: YES), then the process 4000 can proceed with generating a recommendation that includes the course of action 4500. If, however, the predicted beauty outcome is not consistent with the desired beauty outcome 4400 (step 4300: NO), then the process 4000 can proceed with generating a recommendation that does not include the course of action 4550.

In instances where a course of action is not recommended to the individual by the system, the system can compute a new responsiveness metric that relates to a new course of action. This process can be repeated until one or more courses of action that are predicted to be effective can be recommended to the individual by the system. As a non-limiting example, the system can receive a measurement of one or more skin biomarkers that correspond to responsiveness to a retinol treatment, and if the responsiveness metric is positive or above a minimum threshold indicative that the treatment would be effective, the system can recommend the retinol treatment to the individual. If, however, the responsiveness metric is negative or below a minimum threshold indicative of good responsiveness to the treatment, the system can expressly recommend not proceeding with the treatment and/or can proceed with another iteration of computation of the responsiveness metric, for another treatment, until one or more recommendations are given to the individual.

One or more thresholds can be static or fixed for an individual or given set of inputs or biomarker measurements, or can be dynamic or fluid in response to updated information about responsiveness to a particular treatment for an individual or group of individuals, or for a variable or group of variables or inputs of the system. In addition, thresholds can be associated with predicted positive responses or predicted negative responses to a particular treatment. For example, in embodiments, a first threshold of the measurement corresponds with a positive responsiveness metric and predicted responsiveness of skin of the individual to the course of action, and a second threshold of the measurement corresponds with a negative responsiveness metric and predicted non-responsiveness of skin of the individual to the course of action.

While a course of action recommended (or not recommended) by a system can include a particular skincare treatment or routine, any course of action that can have a direct or indirect effect on skin health can be implemented as a course of action in various embodiments. For example, in embodiments, the course of action comprises a lifestyle change (e.g., drinking enough water), a cessation of a negative habit (e.g., excessive nicotine use, excessive alcohol use, or the like), a reinforcement of a positive habit (e.g., adequate sleep, diet, exercise), a selection of a product (e.g., skincare product), a selection of a service (e.g., massage, facial, light therapy), an application of a product to skin of the individual, or any combination thereof. As such, the course of action can encompass any of a variety of possible actions and is not necessarily limited to a product recommendation.

In embodiments, the desired beauty outcome is computed as an assumed desired beauty outcome for the individual based on one or more characteristics of the individual. For example, if the system accepts as an input information about the individual's chronological (i.e., actual) and apparent (i.e., presenting) age, and if the individual appears older than they actually are, the system might assume the individual is desirous of lowering their apparent age, for example, based on desired beauty outcomes of other individuals with the same or similar inputs. However, in embodiments, the desired beauty outcome is computed as an expressed desired beauty outcome based on one or more inputs provided by the individual. For example, if the individual appears older than they actually are, but the individual provides an input to the system communicating that they are satisfied with their current apparent age, the desired beauty outcome may not involve any changes to the individual's apparent age and may focus on other aspects of a desired beauty outcome.

In embodiments, the measurement of the one or more skin biomarkers of the individual is transmitted from a measurement device and received by a computational device, either or each of which can be an element of the system. In embodiments, the measurement device comprises an on-site biomarker reader configured for on-site measurement of the one or more skin biomarkers of the individual. In embodiments, the computational device comprises a networked server, a cloud computing system, a smartphone, a tablet, a desktop computer, a laptop computer, a smart watch, a wearable device, or the like, or any combination thereof.

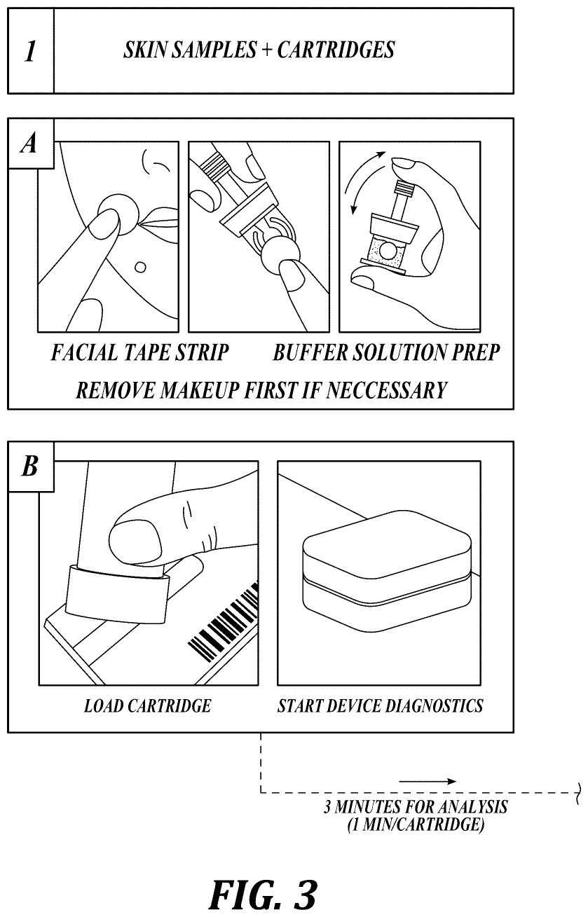

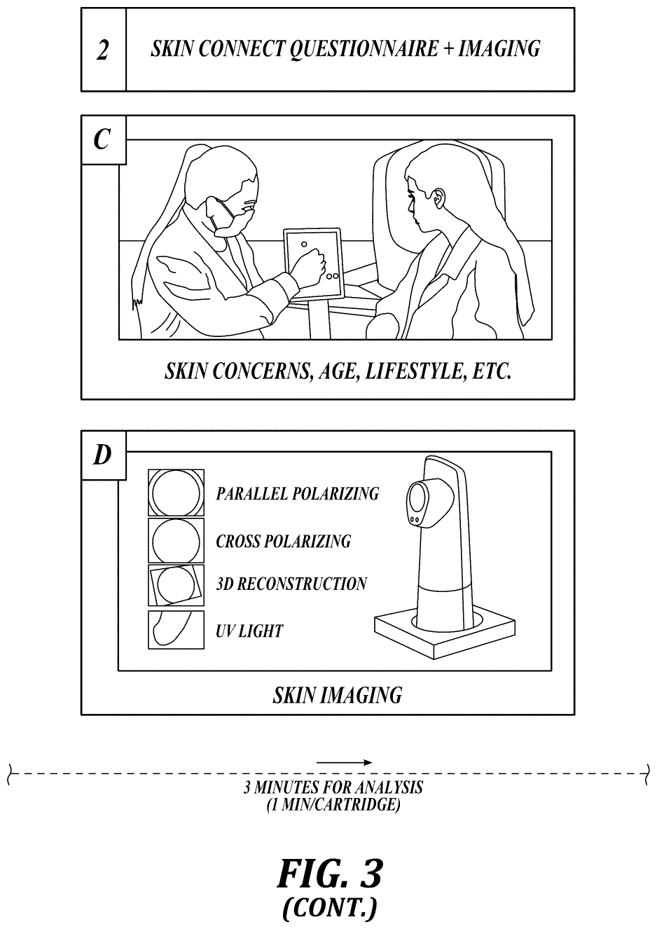

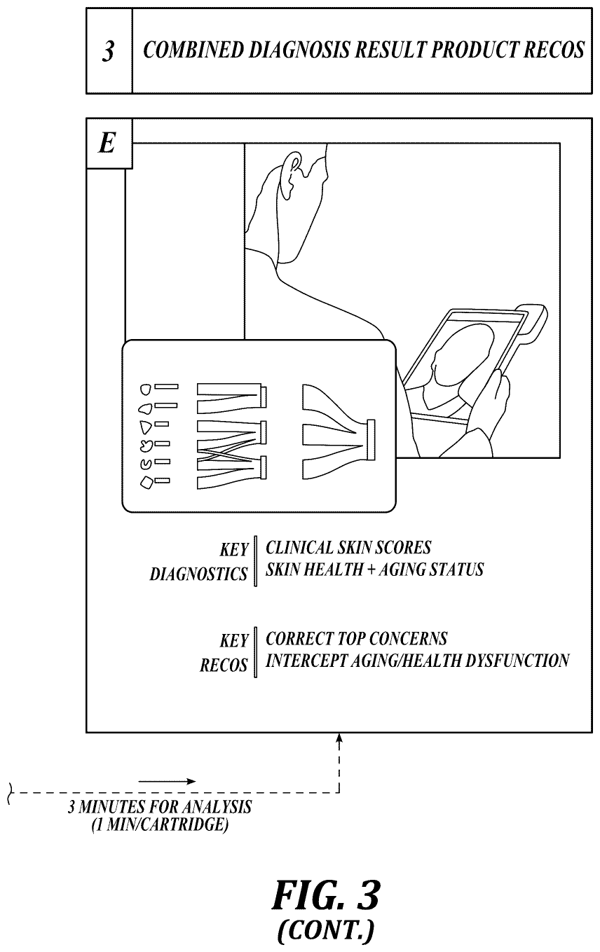

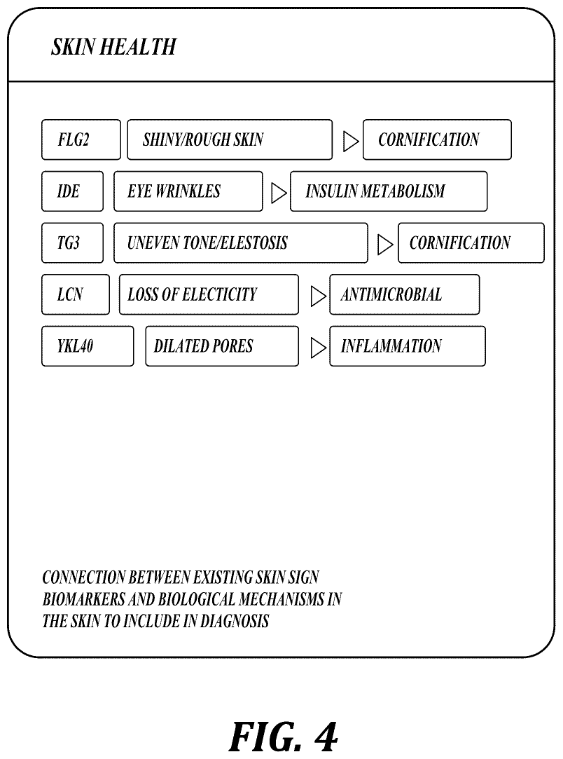

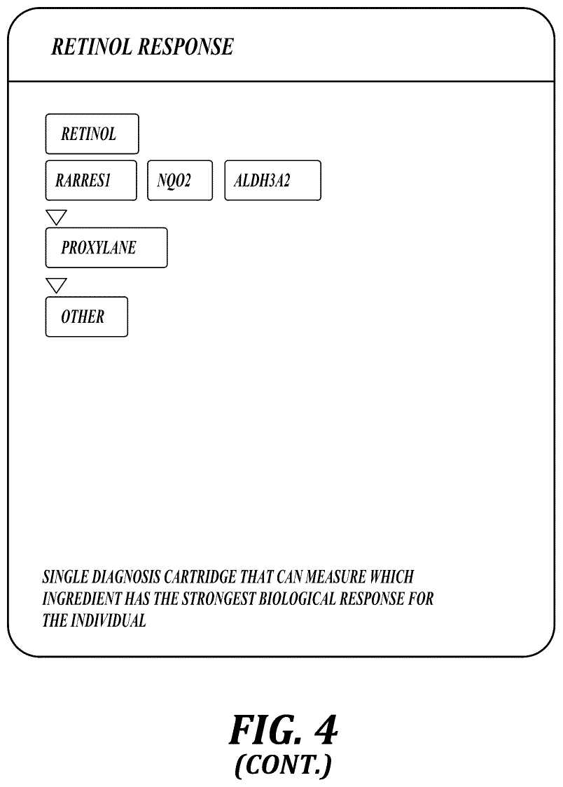

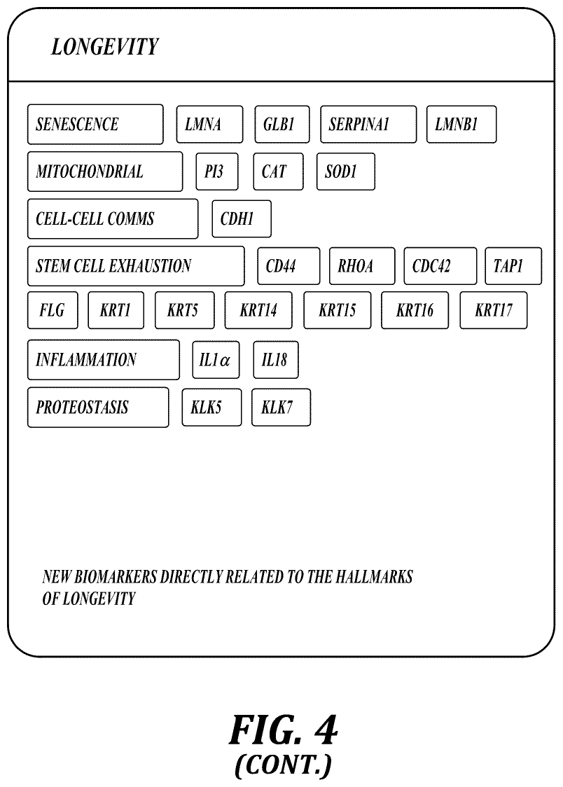

FIG. 3 shows examples of steps for use of a system for management of skin health, including with generation of a recommended course of action, and FIG. 4 shows examples of skin biomarkers and skin health characteristics thereof, retinol response skin biomarkers, and longevity skin biomarkers, according to aspects of the disclosure.

As a first step, skin samples can be captured using facial tape strips, and exposed to buffer solution as part of a sample test preparation procedure. If an individual is wearing makeup, which could interfere with sample collection from the skin surface, the makeup can be removed before sample collection. Once the sample is captured with a cartridge, the cartridge is loaded into a measurement device which then proceeds with measurement of one or more biomarkers. As a non-limiting example, an enzyme-linked immunosorbent assay (ELISA) based measurement device can be used. An example of such an ELISA based measurement device includes the FREND® instrument (NanoEnTek®), however, alternative ELISA based measurement devices can be implemented in various embodiments without departing from the scope and spirit of the disclosure. In at least some embodiments, an ELISA assay can be used for measurement of one or more skin biomarkers, without necessarily relying on any particular measurement device or platform.

In embodiments, an individual can be presented with a questionnaire configured to capture input regarding the individual's health, lifestyle, skincare concerns, age, perceived age, or the like. In embodiments, skin imaging can be implemented to capture imagery of the skin of the individual, and that imagery further processed for generation of one or more inputs in combination with biomarker measurement information. In various embodiments, skin biomarker measurement data, optionally in combination with additional data (e.g., biomarker measurements, skin imagery, skin biomechanics, and the like), can be used to train a machine learning (ML) model for prediction of responsiveness to a skincare treatment based on past responsiveness of one or more individuals with the same or similar ML input data to the skincare treatment.

In embodiments, a system comprises circuitry configured to receive an imagery measurement of skin of the individual, an electromechanical measurement of skin of the individual, or both, and circuitry configured to compute the responsiveness metric based on the imagery measurement, the electromechanical measurement, or both. In embodiments, the imagery measurement is transmitted from an image capture device and received by a computational device, and wherein the electromechanical measurement is transmitted from an electromechanical device and received by the computational device.

In embodiments, a system comprises circuitry configured to receive a plurality of measurements of a plurality of skin biomarkers of the individual and compute, based on the plurality of measurements, the predicted beauty outcome that is predicted to occur due to the course of action. In embodiments, the circuitry computes the predicted beauty outcome based on measurements of one or more relatively up-regulated skin biomarkers, measurements of one or more relatively down-regulated skin biomarkers, or any combination thereof.

In embodiments, the responsiveness metric is computed based at least in part on: a skin health characteristic of skin of the individual selected from the group consisting of: a skin texture, a cornification level, a presence or absence of an eye wrinkle, a level of insulin metabolism, a level of skin evenness or elastosis, a degree of loss of elasticity of skin, an antimicrobial property, a presence or absence of a dilated pore, a degree of inflammation, and any combination thereof; a predicted response of skin of the individual to a retinol treatment selected from the group including, but not necessarily limited to: retinol (vitamin A₁), C-beta-D-xylopyranoside-2-hydroxy-propane (C-Xyloside or ProXylane®), azelaic acid, and any combination thereof; a longevity characteristic of the individual selected from the group consisting of: a degree of inflammation, a level of stem cell exhaustion, a degree of DNA instability, a degree of proteostasis, a degree of epigenetic change, a level of microbiome change, a level of mitochondrial function, a level of cellular communication, a level of cellular senescence, and any combination thereof.

In embodiments, the responsiveness metric is computed based at least in part on a skin health characteristic of skin of the individual and the one or more skin biomarkers comprises a protein selected from the group including, but not necessarily limited to: filaggrin 2 (FLG2), Insulin-degrading enzyme (IDE), epidermal transglutaminase (TG3), lipocalin-1 (LCN1), Chitinase-3-like protein 1 (YKL40), and any combination thereof.

In embodiments, the responsiveness metric is computed based at least in part on a predicted response of skin of the individual to a retinol treatment and the one or more skin biomarkers comprises a protein selected from the group including, but not necessarily limited to: Retinoic acid receptor responder protein 1 (RARRES1), Aldehyde dehydrogenase family 3 member A2 (ALDH3A2), N-ribosyldihydronicotinamide:quinone dehydrogenase 2 (NQO2), and any combination thereof.

In embodiments, the responsiveness metric is computed based at least in part on a longevity characteristic of the individual and the one or more skin biomarkers comprises a protein selected from the group including, but not necessarily limited to: lamin A/C (LMNA), galactosidase beta 1 (GLB1), serpin family A member 1 (SERPINA1), lamin B1 (LMNB1), peptidase inhibitor 3 (PI3), catalase (CAT), superoxide dismutase 1 (SOD1), cadherin 1 (CDH1), CD44 molecule (IN blood group) (CD44), ras homolog family member A (RHOA), cell division cycle 42 (CDC42), transporter 1, ATP binding cassette subfamily B member (TAP1), filaggrin (FLG), keratin 1 (KRT1), one or more keratin genes (KRTs), keratin 14 (KRT14), keratin 15 (KRT15), keratin 16 (KRT16), keratin 17 (KRT17), interleukin-1 alpha (IL-1α), interleukin-18 (IL-18), Kallikrein Related Peptidase 5 (KLK5), Kallikrein Related Peptidase 7 (KLK7), and any combination thereof.

### Biomolecule Extraction Devices and Methods

As used herein, the term “biomolecule extraction device” includes, but is not necessarily limited to, the devices as described in U.S. Pat. No. 10,520,404 B2, the contents of which are incorporated by reference herein in their entirety for all purposes.

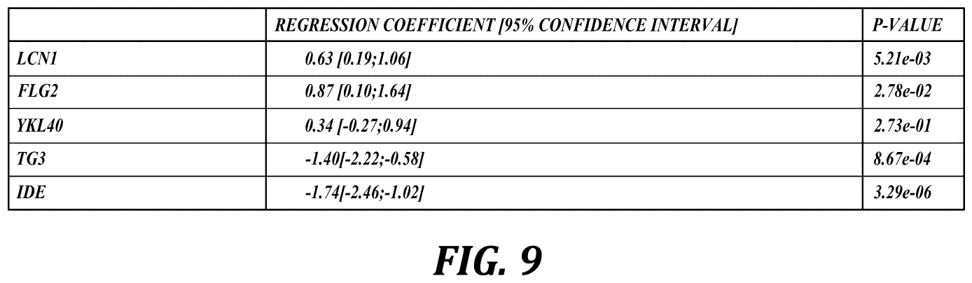

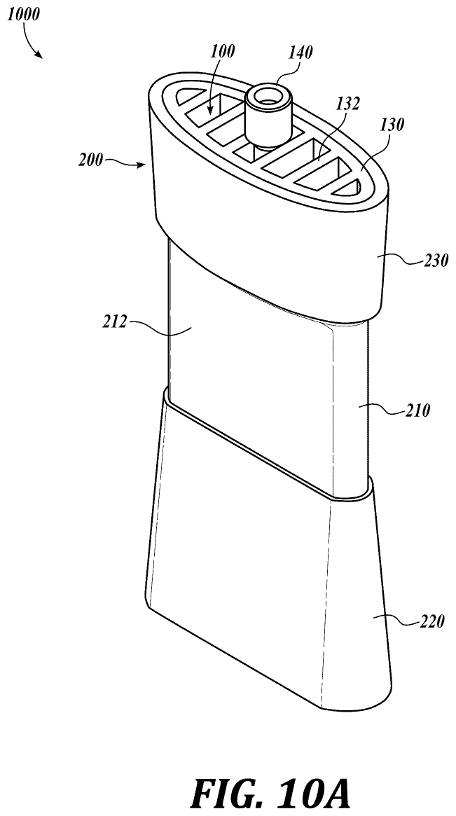

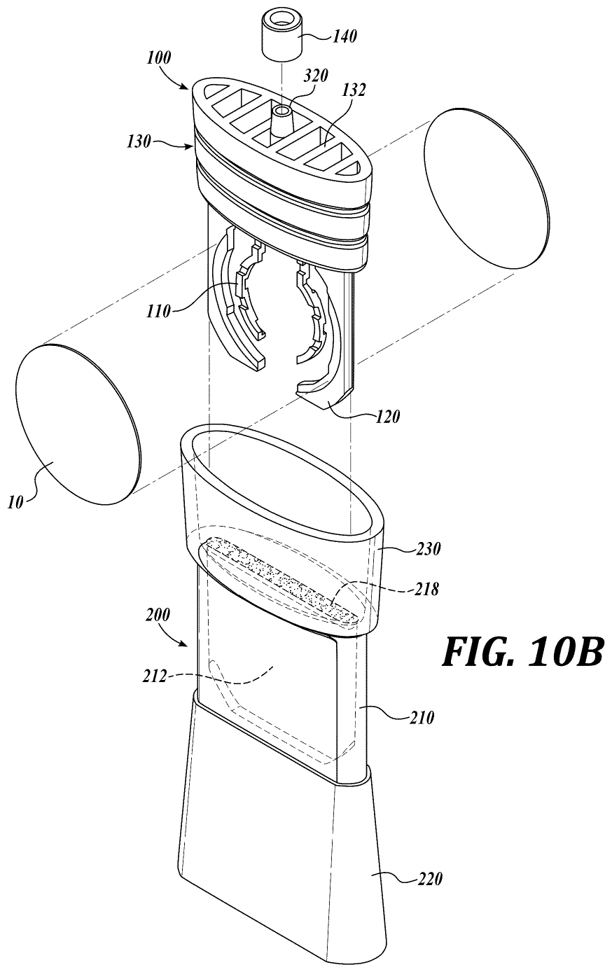

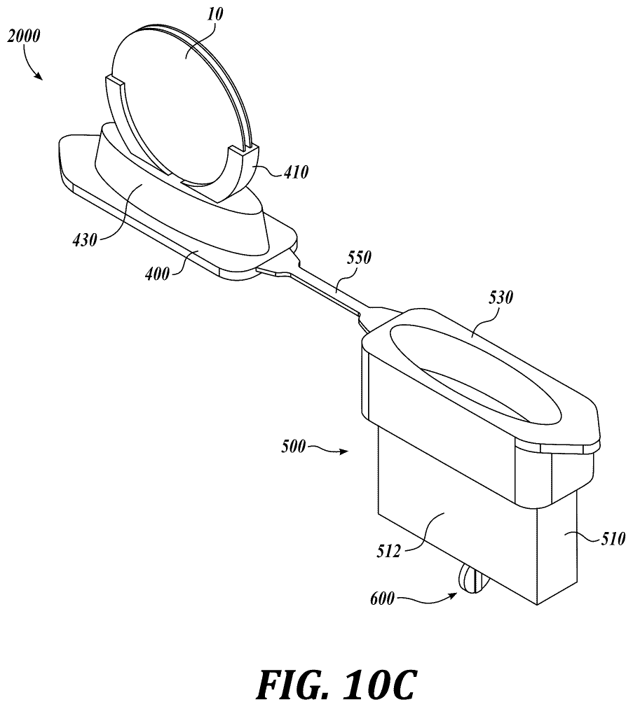

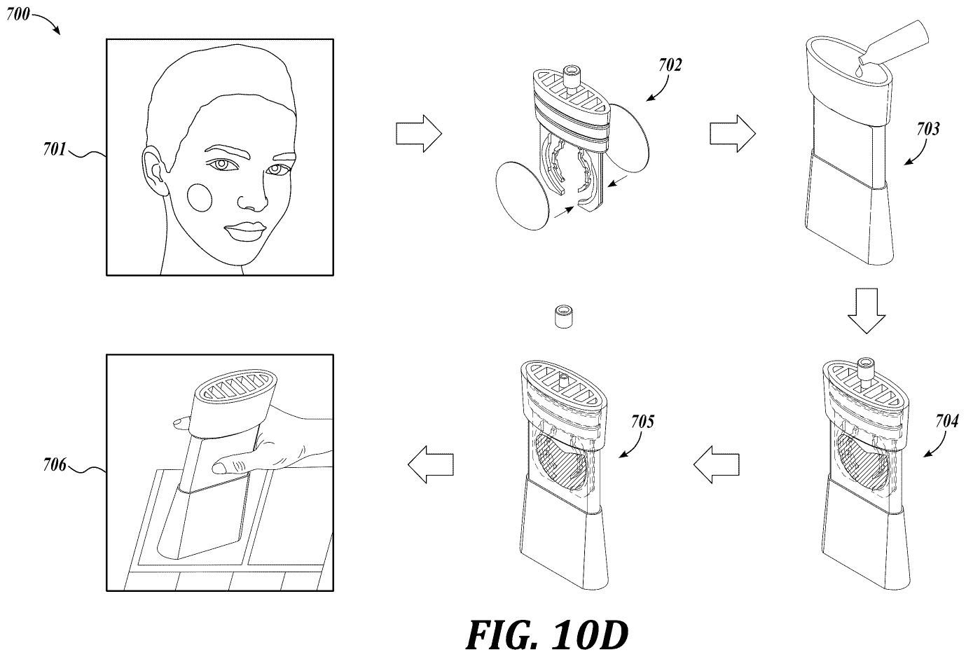

As shown at FIGS. 10A-10D, an example biomolecule extraction device 1000 can comprise an insertion part 100 to which a sample containing collected biomass is fixed, a body part 200 which receives a lysis buffer inside and into which the insertion part 100 is inserted to extract biomolecule from the collected biomass, and at least one discharge part provided at the insertion part 100 or the body part 200.

The insertion part 100 can comprise at least one fixing part (110, 120) to which the sample is fixed. The fixing part (110, 120) can fix the sample by adhesive bonding or physical bonding, and the sample collecting biomass may be properly applied, for example, to a tape or membrane, or the like, having an adhesive surface depending on the collection method.

In case the fixing part (110, 120) fixes the sample by adhesive bonding, a tape with an adhesive surface, for example, a tape for obtaining samples 10 having an adhesive surface coated with an adhesive substance is used and bonded to the fixing part using the adhesiveness left on the tape after collecting sample. Alternatively, it may be configured to apply the adhesive substance to the fixing part (110, 120) and fix a non-adhesive sample.

In case the fixing part (110, 120) fixes the sample by physical bonding, it can be fixed by insertion. For example, a tape for obtaining samples 10 can be used, and the insertion part 100 can comprise the fixing part (110, 120) adhered by having at least part of the tape for obtaining samples 10 in contact.

Here, the fixing part (110, 120) can comprise a first fixing part 110 formed on the inner side and a second fixing part 120 formed on the outer side of the first fixing part 110. The fixing part can comprise two of each of the first fixing part 110 and the second fixing part 120 on the same plane in a form extended perpendicularly downwards.

Also, the two tapes for obtaining samples 10 can be adhered to face each other with the first fixing part 110 and second fixing part 120 interposed. The tape for obtaining samples 10 can be in a disk form.

One side of the tape for obtaining samples 10 can be an adhesive surface and the other side can be a non-adhesive surface. If the adhesive surface of the tape for obtaining samples 10 is attached to and then detached from the subject's skin, skin tissue comprising dead skin cells is collected by being attached to the adhesive surface, and protein can be extracted using this.

The two tapes for obtaining samples 10 containing collected skin tissue as above may be attached and fixed so that the adhesive surfaces face each other as separated at a certain interval with the first fixing part 110 and second fixing part 120 interposed. Here, since the tapes for obtaining samples 10 are attached to be separated at a certain interval with the first fixing part 110 and second fixing part 120 interposed, and this prevents the tapes for obtaining samples 10 from being adhered to each other.

The two tapes for obtaining samples 10 are separated from each other as much as the thickness of the first fixing part 110 and second fixing part 120. When the insertion part 10 is inserted into the body part 200, a lysis buffer 20 fills in the space between the tapes for obtaining samples 10.

Thus, the thickness of the fixing parts (110, 120) becomes a separation distance between the two tapes for obtaining samples 10 and also a parameter for determining the volume of the lysis buffer to be introduced.

Here, the first fixing part 110 and the second fixing part 120 can have the same thickness or the first fixing part 110 positioned on the inner side may have a thickness thinner than that of the second fixing part 120. In case a distance spaced enough not to be adhered to each other due to the rigidity of the tapes 10 is maintained, the first fixing part 110 can have a minimum thickness or even may be removed. In this case, the two tapes for obtaining samples 10 are spaced apart from each other by the thickness of the second fixing part 120 positioned on the other side. The part of the second fixing part 120 where the tape 10 is fixed has a size slightly greater than the outer circumference of the tape 10, and is engraved by the thickness of the tape, so that the insertion part 100 is not caught by protrusions of the tapes 10 or dead region is not generated when the insertion part 100 is inserted into the chamber 210 after the tapes 10 are fixed. Further, the first fixing part 110 positioned on the inner side prevents the tapes for obtaining samples 10 from being adhered to each other in their middle parts.

The first fixing part 110 and the second fixing part 120 can be made into a linear bar shape having a predetermined thickness, but in an aspect, at least part of it is in a bent curved shape. By configuring part of the first fixing part 110 and the second fixing part 120 to be in a curved shape, a user can apply pressure without interference by the first fixing part 110 or the second fixing part 120 when applying pressure to a pressing part 212. The structures of bumps are added to the first fixing part 110 as necessary, which enable to minimize the contact surface of the tapes for obtaining samples 10 and maximize the surface area of the tapes for obtaining samples 10 exposed to the lysis buffer 20, and allow free movement of the lysis buffer 20.

The body part 200 can comprise a chamber 210 which forms a predetermined space inside to receive the lysis buffer 20 and into which the tapes for obtaining samples 10 are inserted to be immerged in the lysis buffer 20.

A base 220 is provided in the lower part of the chamber 210 to support and stand the chamber 210. The base 220 can be formed with the slope such that the cross-sectional area increases downward in order for the body part 200 to stand stably.

The thickness of the inside space of the chamber 210 is preferably configured such that each of non-contact surfaces 14 of the two tapes for obtaining samples 10 is adhered to the inner wall of the chamber 210 when the two tapes for obtaining samples 10 are inserted. Accordingly, most of the lysis buffer 20 contained in the chamber 210 fills the space between the two tapes for obtaining samples 10.

Such configuration is able to minimize dead space and to maximize the contact surface with the tapes for obtaining samples 10, which results in increasing the extraction concentration of biomolecule to a level which can be measured with a small amount of sample, even with applying a minimum amount of the lysis buffer 20.

In particular, conventionally, since skin tissue samples adhered to the tapes for obtaining samples 10 are hydrophobic and thus the lysis buffer 20 did not spontaneously spread out, external force should be compulsorily applied or additional operation should be performed by a device. Also, in order to avoid a problem that the tapes 10 rise up when immerged, external force should be consistently applied to the tapes 10.

Also, as explained above, the biomolecule extraction device 1000, which makes the contact surface with the tapes for obtaining samples 10 relatively very large with respective to the volume of the lysis buffer 20, can dissolve cells by being shaken once or twice and being left to stand. When the insertion part 100 is inserted into and joined with the body part 200, strong fixing force can be maintained, and thus the lysis buffer 20 can fill the empty space between the tapes without additional compulsory external force or additional operation. The condition for contact can be effectively maintained. Thereby, the efficiency of extracting proteins can be rapidly increased.

In the cases of applying conventional Petri dish and Eppendorf tube, the SA:V ratios do not exceed 0.4. By comparison, in the case of applying the device, since the cross-sectional area of the tapes 10 is 380 mm² and the amount of the lysis buffer 20 to be introduced is 200 μL when using a product such as D-Squame disk manufactured by Cuderm Corporation, the calculated SA:V ratio is 1.9, which is over 0.4, but the SA:V ratio practically exceeds 1.4 in order to secure tolerance during the manufacture of injection molding of an extractor and spare space of a mold, for example. The SA:V ratio can be further increased by reducing a distance between the tapes for obtaining samples 10. However, considering that a target discharge amount for one time is 35-40 μL and a substantial minimum gap that can be pressed when discharging is about 100 μm, while preventing the adhesion between the two tapes 10, the SA:V ratio can be raised up to about 9.15. As such, the SA:V ratio is relatively high, which enables to extract proteins with a minimum amount of samples and also raise the extract concentration.

Meanwhile, the entrance of the chamber 210 can be left open or comprise a shielding film 218. In case the entrance is left open, a user introduces the lysis buffer 20 into the chamber 210 before inserting the insertion part 100; in case the entrance comprises the shielding film 218, a predetermined amount of the lysis buffer 20 can be contained inside the chamber 210 in advance.

The shielding film 218 can be comprised of, for example, aluminum foil or vinyl film, or the like, to seal the entrance of the chamber 210, and the user can insert the insertion part 100 after removing the shielding film 218, or push the insertion part 100 to penetrate the lysis buffer 218 so as to be inserted into the chamber 210. The position of the shielding film 218 is not fixed to the entrance of the chamber 21 but can be in the introduction part 230 as necessary.

An introduction part 230 can be comprised on the upper part of the chamber 210. The introduction part 230 can be configured to extend upward from the entrance of the chamber 210 by a certain height. Further, a sealing part 130 sealing the chamber 210 can be provided at one end of the insertion part 100, which is closely contacted to the inner wall of the introduction part 230 so as to correspond to the introduction part 230.

That is, the introduction part 230 is open upward, and when the tapes for obtaining samples 10 attached to the first fixing part 110 and the second fixing part 120 are inserted into the chamber 210, the sealing part 130 is fitted in the introduction part 230 to seal the chamber 210.

Further, if the introduction part 230 and the sealing part 130 are cut horizontally, the cross sections thereof may be shown in an oval shape. If the cross sections thereof are to be in a square shape, the adhesion force is not distributed evenly, and thus the sample may leak from the corners; and if the cross sections are made a circular shape, the adhesion force is distributed evenly, but they have a bulky volume. Thus, in embodiments, the cross sections of the introduction part 230 and sealing part 30 are in an oval shape. Here, the sealing part 130 can comprise a plurality of reinforcing ribs 132 for reinforcing the strength thereof. The reinforcing ribs 132 boost the adhering force and fixing force of the sealing part 130 to the inner wall of the introduction part 230 while reinforcing the strength so that the sealing part 130 is not deformed when being joined with the body part 200.

Meanwhile, the biomolecule extraction device 1000 can comprise at least one discharge part 300 equipped in the insertion part 100 or body part 200. In embodiments the discharge part 300 is provided to the insertion part.

Here, the discharge part comprises a discharge flow path which penetrates the sealing part and allows the chamber 210 to communicate with the outside and discharge the sample, and an expanded discharge port 320 which is provided at the end of the discharge flow path and has an inner diameter greater than the inner diameter of the discharge flow path so as to constantly discharge the sample.

The discharge flow path communicates with the chamber 210 at one end and is connected to the expanded discharge port 320 at the other end, through which a sample having the protein extraction can be discharged. The discharge flow path should be designed to have a minimum inner diameter before reaching the expended discharge port, in order to minimize the dead space and increase the total discharge amount. In embodiments, the discharge flow path has a diameter of 850 μm, which is half of that of the expanded discharge port 320.

The amount of the discharged sample can be adjusted to a constant amount by adjusting the size of the inner diameter of the expanded discharge port 320.

A detachable sealing cap 140 can be additionally provided to the expanded discharge port 320, which prevents the sample from being randomly discharged and ensures the user's safety.

Meanwhile, a pressing part 212, which is formed on the outer wall of the chamber 210 such that external force can be applied when the user discharges the sample, and presses the sample, may be provided. Basically, a region corresponding to a hollow part the curved shape of the above-described first fixing part 110 forms in the outer wall of the chamber 210 forms a pressing part 212.

Specifically, the user applies pressure to the pressing part 212 to discharge the extracted sample, and when applying pressure, the inner walls of the chamber 210 are bent in a parabolic shape and thereby the extracted sample is squeezed out of the outlet.

FIG. 10D shows a process diagram illustrating an example process of extracting protein using a biomolecule extraction device, according to aspects of the disclosure. First, tapes for obtaining samples 10 are attached to and then detached from the subject's skin to take the skin tissue. Then, the tapes are adhered such that they face each other with the first fixing part 110 and the second fixing part 120 interposed.

Then, the lysis buffer 20 is introduced into the chamber 210 of the body part 200 and the insertion part 100 is inserted into the body part 200. Here, the lysis buffer 20 is contained in the chamber 210 in advance and may be provided in a sealed state by the shielding film 218.

Thereafter, the biomolecule extraction device 1000 is shaken once or twice with the tapes for obtaining samples 10 positioned inside the chamber 210 and is left to stand for 1 minute or several minutes as necessary. In this process, the cells are dissolved in the lysis buffer 20 and the proteins are extracted.

Then, after removing the sealing cap 140, the pressing part is pressed to discharge the sample having the extracted proteins into the inlet, and the test through antibody response proceeds.

FIG. 10C shows a perspective view of an example biomolecule extraction device, according to aspects of the disclosure. A biomolecule extraction device 2000 can be made by comprising an insertion part 400, a body part 500 and a discharge part 600, briefly.

Here, the discharge part 600 can be equipped in the body part 500, not in the insertion part 400. The insertion part 400 comprises a fixing part 410, and the tapes for obtaining samples 10 can be physically joined with the fixing part 410 by means of insertion or be adhered and fixed thereto by applying an adhesive.

One aspect suggests a case where two tapes for obtaining samples 10 are fixed to the fixing part 410, but it is also possible to fix and use a single or three or more tapes for obtaining samples 10, as necessary.

The insertion part 400 and the body part 500 can be connected to each other by a connection part 550. The body part 500 comprises an introduction part 530, and the insert part 400 can comprise a sealing part 430 to correspond to the introduction part.

Meanwhile, the body part 500 can comprise a chamber 510 which contains the lysis buffer, and the tapes for obtaining samples 10 which are fixed to the fixing part 410 can be inserted into the chamber 510 and the biomolecule can be extracted by the lysis buffer contained in the chamber 510 as the insertion part 400 is inserted into the body part 500.

Once the biomolecule extraction is completed, the user presses a pressing part 512 formed on the outer wall of the chamber 510 to discharge the extracted sample discharge through a discharge part 600. Here, various modifications including as described herein can be equally applied to the pressing part 512.

The biomolecule extraction device has the following effects.

First, the biomolecule extraction device can perform the whole process of fixing a sample and injecting a buffer, assembling the insertion part and mixing, letting stand still and extraction, and the like, within 5 minutes, which results in innovatively reducing the time to be taken, whereas most of the conventional protein or nucleic acid extraction methods required at least 20 to 30 minutes in total for mechanical or physical impact application, repetitive centrifugation, filtering and other process, in order to break intercellular bonding or cell membrane.

Second, the biomolecule extraction device is performed in a non-impact and non-power manner, which needs neither broad experimental space nor a complicated system, ensures the user's safety, and is able to avoid harmful effect due to wastes because an extremely small amount of buffer is applied for a single use.

Third, the biomolecule extraction device can minimize the dead space, greatly increase the surface area:volume ratio, which allows the protein extraction without external force in a high concentration, and constantly discharge after extraction. Thus, a precise test with a test kit is possible.

Fourth, the biomolecule extraction device simplifies the protein extraction process in cells and can be independently used as one device without the need of additional devices, such as a centrifuge, a pipet, and can be used without the user's skill. Thus, the user's convenience can be enhanced.

The disclosed approaches can collect biomarker samples from the skin tissues, but a person skilled in the art can variously modify and change the devices and approaches without deviating from the scope and spirit of the disclosure. The extraction device can be used for extracting from diverse biomass various biomolecules available for various analysis and diagnosis. The application scope includes biotechnology, molecular biology, medical science, pharmaceuticals, cosmetics, genetic engineering, diagnosis, health care, and the like, but is not limited thereto.

### Terminology

Descriptions of terms in this section are intended to facilitate an understanding of aspects and embodiments of the disclosure and do not necessarily exclude the meanings of these terms as they may be ordinarily used in the art. Wherever a conflict arises between a meaning of a term in the art and a meaning of the term in this disclosure, the meaning of the term in this disclosure shall prevail.

As used herein, including in the claims, the terms “a,” “an,” “the,” and the like, refer to the singular and the plural forms of the object or element referenced. The term “comprising” is inclusive or open-ended and does not exclude additional, unrecited elements or steps. The term “consisting of,” as used in a claim, excludes any element, step, or ingredient not specified in the claim. The term “consisting essentially of,” as used in a claim, limits the scope of the claim to the specified materials or steps and those that do not materially affect the basic and novel characteristic(s) of the claim.

As used herein, the term “device,” and similar terms, refers to an electronic computational device that is configured, or that can be configured, to perform all or part of a method or process of the disclosure (see, e.g., computing device 3500 of FIG. 11). The term “system,” and similar terms, refers to two or more, i.e., multiple, devices that together are configured, or can be configured, to perform all or part of a method or process of the disclosure. Devices and systems can be operably connected by one or more electronic communication connections (e.g., wired or wireless, e.g., Bluetooth®, Wifi®, infrared (IR), or the like), or network connections (e.g., intranet, internet, telecommunications network, or the like) for electronic communications that utilize one or more communication protocols for the transmission or exchange of data, for example, for carrying out all or part of a method as a computational method. The term “computational method” refers to a method, or a portion of a method, that is carried out substantially or entirely by a device or system, optionally with little or no human or manual input.

As used herein, the term “instructional material,” and similar terms, refers to any material or media, whether print, audio, visual, illustrative, audiovisual, tactile, or otherwise, that can convey to an individual information about how to use a kit, device, system, or other aspect of the disclosure in a method or process for skin biomarker measurement.

As used herein, the term “circuitry” refers to one or more physical, computational, electrical circuits configured to perform all or a portion of a method or process of the disclosure. Circuitry can include a processor or microprocessor configured to execute processor-executable instructions, such as software and/or firmware, for enablement of a computer program or software application that accepts inputs as data (e.g., biomarker measurement data, information about a desired beauty outcome, information about one or more possible courses of action) and computes outputs as data (e.g., responsiveness metric, recommendations, and the like). Circuitry can include, instead of or in addition to a processor or microprocessor, dedicated hardware circuitry configured to perform logic operations by way of a configuration thereof.

As used herein, the term “biomarker” refers to a biomolecule that can be quantitatively measured to produce a measurement that meaningfully corresponds to the skin health of an individual. The term “biomolecule” includes, but is not necessarily limited to, proteins, messenger RNAs (mRNAs), lipids, micro-organisms, metabolites, glycans, epigenetic states or changes, DNA, and the like. The term “skin health” refers to one or more underlying biomedical factors that contribute to a healthy state, aging state, or disease state of the skin; such factors include, but are not limited to, metabolism, hydration, ultraviolet (UV) damage, senescence, mitochondrial activity, cellular signaling, stem cell status or exhaustion, inflammation, proteostasis, insulin metabolism, antimicrobial activity, and the like. The term “skin health” also refers to one or more visible or macro-observable factors that can correspond to a healthy state, aging state, or disease state of the skin; such factors include, but are not limited to, dry skin, wrinkles, crow's feet, elastosis, cheek folds, dischromia, pigmentation, brightness, papyraceus aspect, elasticity, oiliness, shininess, roughness, scaliness, erythrosis, pore size or visibility, and the like. Accordingly, the term “skin health” is inclusive and refers to underlying skin health factors as well as macroscopic or plainly observable skin health factors.

As used herein, the term “measurement device” refers to a device that is configured to measure one or more skin biomarkers on-site, or at a point of care (POC), to produce a measurement. Non-limiting examples of measurement devices include various devices, systems, kits, and apparatuses for in vitro quantitation of biomarkers, such as microfluidic immunofluorescence analyzers (e.g., FREND™ system as provided by NanoEntek™), and the like.

As used herein, the term “predicted beauty outcome” refers to a skin health of an individual that is computed, by a system of the disclosure, as being likely to occur as a result of a course of action that is taken by the individual. The predicted beauty outcome can be computed based on one or more biomarker measurements, a chronological or actual age of the individual, a clinical or presenting or apparent age of the individual, a genetic or other inheritable feature of the individual (e.g., a genetic allele, an epigenetic feature, and the like), an environmental feature of the individual (e.g., place of home, place of work), lifestyle features (e.g., smoking or drinking status, time spent outdoors, and the like), or any combination thereof.

As used herein, the term “desired beauty outcome” refers to a skin health of an individual that is sought after by the individual or that is predicted, by a system of the disclosure, as being sought after by the individual (or that is assumed, by the system, as being sought after by the individual), based on one or more characteristics of the individual that are consistent with one or more characteristics of one or more other individuals. For example, if the system comprises a correlation between individuals that are between 40 and 80 years of age and the desire to reduce the appearance of crow's feet, and the individual is between 40 and 80 years of age, the system may assume that the individual also desires to reduce the appearance of crow's feet as the desired beauty outcome; however, alternatively, the individual can provide, as a manual input, the desired beauty outcome.

As used herein, the term “course of action” refers to an activity, such as the selection or purchase of a skincare product (e.g., a retinol-based skincare product), the application of the skincare product, or the like, that can be taken by an individual to try to improve their skin health. The term “course of action” can also include lifestyle changes, such as changes in smoking or alcohol consumption, time spent in the sun, use of sunscreen, consumption of water, sleep or rest time, time spent with electronic media, and the like.

As used herein, the term “responsiveness metric” refers to a quantitative metric that represents the likelihood of the skin health of an individual improving or advancing toward the desired skin health as a result of a particular course of action. The responsiveness metric can be represented in any numerical format, including integers, decimals, percentages, ratios, binary (positive or negative), and the like. As used herein, a threshold can be applied by a system of the disclosure to a measurement of a biomarker; if the measurement surpasses the threshold, the responsiveness metric can be positive or indicative of a higher likelihood of the skin health of the individual as improving, and if the measurement does not surpass the threshold, the responsiveness metric can be negative or indicative of a lower likelihood of the skin health of the individual as improving. Computation of the responsiveness metric can be performed by a system of the disclosure based on one or more biomarker measurements, an imagery measurement of the skin or face of the individual (e.g., tone, coloration, lines or wrinkles, blemishes, or the like), an electromechanical measurement of the skin or face of the individual (e.g., softness, firmness, elasticity, pore size, conductivity, hydration level, or the like), or any combination thereof. The imagery measurement can be a result of a computer vision analysis of electronic imagery of the skin or face of the individual. Computer vision analysis of the skin or face can implement a computer vision algorithm that analyzes the imagery, computationally detects features, and computes at least part of the imagery measurement based on the detected features.

As used herein, the term “recommend” as used in the phrase “circuitry configured to recommend or not recommend” refers to a presentation, by the circuitry (or a device or system comprising the circuitry), of one or more courses of action that can be taken, by a user or individual, to try to advance their skin health toward a desired beauty outcome or a desired skin health. The presentation can occur by way of a visual or audiovisual message on a graphical user interface (GUI) of a device, an advertisement (e.g., a personalized ad), a social media platform, a software application of a computational device (e.g., a L′Oreal mobile app as can be downloaded to a mobile device from an app store), or the like.

### EXAMPLES

Example 1: Clinical vs. chronological skin age: exploring determinants and stratum corneum protein markers of differential skin aging in 351 healthy women. (see, for example, Foucher A, et al. Clinical vs. chronological skin age: exploring determinants and stratum corneum protein markers of differential skin aging in 351 healthy women. Sci Rep. 2024 Oct. 9; 14(1):23643, which is incorporated by reference herein in its entirety for all purposes).

Apparent skin age can be determined by several clinical measurements and can differ from chronological age, hence defining age acceleration/deceleration (Age A/D). Using data from 360 women with dermatological scoring of 21 clinical signs, three well-separated co-occurring classes were identified capturing the dryness, the elasticity and the oily nature of the skin. The risk of each clinical sign was related to the stratum corneum levels of 5 pre-selected proteins, and specific chronological age-adjusted signatures of each clinical sign were identified. Using variable selection approaches, 6 (of the 21) clinical signs which were jointly predictive of chronological age were identified and used to define the clinical skin age, and subsequently age A/D. Applying univariate and multivariate approaches it was found that stratum corneum levels of insulin degrading enzyme (IDE) was protective against (β=−1.74, p=3.3×10⁻⁶; selection proportion>90%) accelerated skin aging. In conclusion, the results support that biomarkers of the stratum corneum can predict skin aging acceleration/deceleration.

### Introduction

The aspect of facial skin is a major determinant of individual clinical age which can diverge from their chronological age, defining the concept of Age acceleration/deceleration (Age A/D). Several studies have reported that facial skin aging could negatively impact individual's self-esteem and overall quality of life. Among all the factors influencing facial skin aging, the exposome which refers to the environmental exposures of an individual throughout their life, plays a role as important as inherited characteristics. Numerous studies involving monozygotic twins have quantified the relative role of genetic background and environmental exposures in both the risk and intensity of facial clinical signs in various populations. A study of 67 monozygotic twin pairs in Japan showed that within-twin-pair differences in skin aging as measured by wrinkle score and facial texture increased with chronological age, hence suggesting a role of life course exposure to environmental factors (including smoking and UV filter application) in Age A/D. A more recent study of 388 pairs of monozygotic twins in South Korea estimated that 43% of the variability of the melanin index could be attributed to genetic variability and the remaining 57% to exposure to environmental factors.

Given the multifactorial aspect of facial skin aging manifestations, the biological mechanisms involved in exposure-triggered Age A/D of the skin remain mainly unknown. However, a recent study of 122 healthy women has established that skin transcriptomic profiles changed with chronological age and was reflective of the clinical skin appearance, hence opening avenues to further explore the predictive value of biological age in Age A/D. Proteomic analysis of the surface of the skin provides a less invasive and closer to the phenotype alternative to transcriptomics. Biomarkers from the stratum corneum have already been related to skin diseases such as Ichthyosis vulgaris, which is associated with loss of function mutation of the filaggrin gene and can be easily measured from D-squame samples. Similarly, many molecular events taking place in the stratum corneum have been revealed via proteomics in lesional and non-lesional skin of patients suffering of Atopic dermatitis. This example uses data from 351 healthy women aged 36 to 75 with expert dermatologist scoring of facial features assessing 21 clinical signs and with D-squame measurement of (N=5) pre-selected protein levels based on prior reported association with chronological age to (i) define homogeneous skin profiles using clustering algorithms and evaluate their relationship with chronological age, (ii) investigate the association between stratum corneum proteins levels and the risk of (groups of) clinical signs, and (iii) to define clinical age as the part of chronological age that could be explained by clinical signs; age A/D as the difference between clinical and chronological age, and explore which proteins are explanatory of age A/D.

### Results

Study population. The study population originally included (N=375) women, aged 36 to 73, (mean values 55.0 years old) as part of two independent studies of volunteers from France and Romania. Each participant underwent in-depth clinical assessment by dermatologist experts to score their clinical features and a D-squame sample on the cheek was collected at recruitment for subsequent targeted proteomic profiling. A total of 15 participants were excluded due to missing information for at least one protein. Score plots along the three first Principal Components (PC)—explaining >80% of the variance in the data—identified two participants with outlying protein profiles along the three first PC. These two participants were excluded from the present analysis leaving (N=351) participants with (N=21) clinical signs and (N=5) proteins assayed. Key characteristics of participants were comparable across study centers, except for participants from Center 1 who were slightly older than in the two other centers.

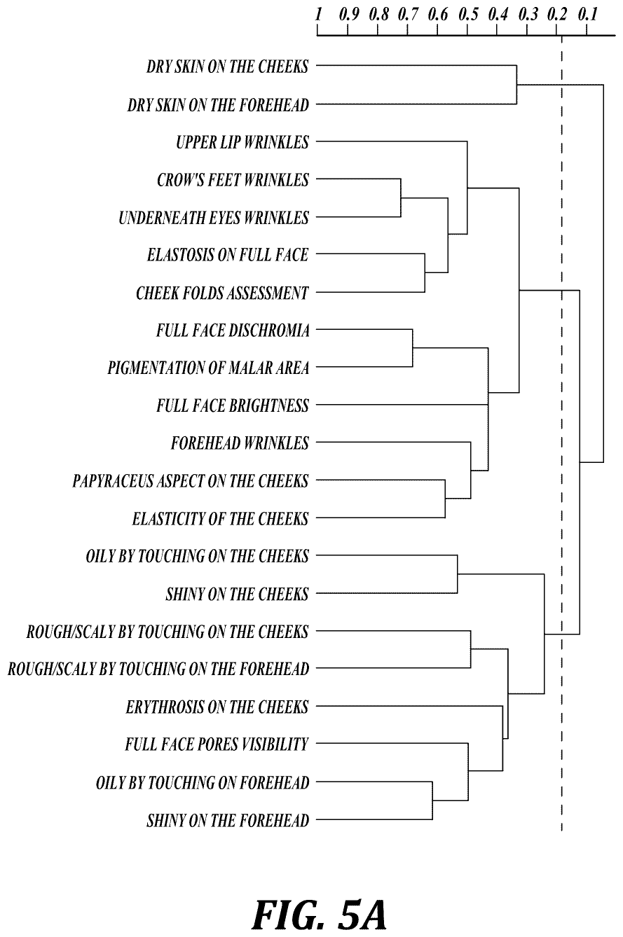

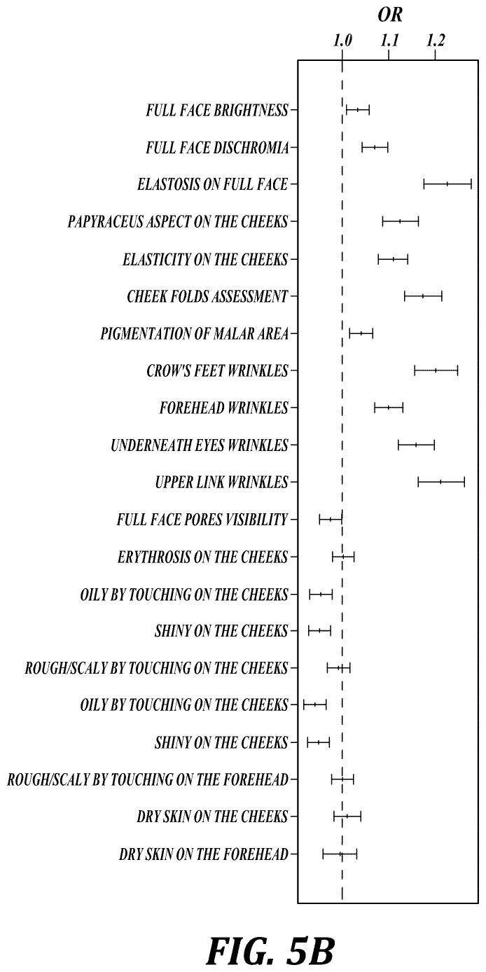

Clinical signs profiling. The prevalence of the 21 clinical signs ranged from 10.5% for dry skin on the forehead to 78.9% for papyraceus aspect on the cheeks in the full population. The prevalence of all clinical signs, except those relating to the oily nature of the skin, was consistently higher in older age group (p<0.0001). Data also supported (more modest) differences in the prevalence of clinical signs across centers with a lower prevalence of most clinical signs in center 1. Hierarchical clustering calibrated to maximize the decrease in within cluster Jaccard's index identified 3 clusters of (co-occurring) clinical signs (FIG. 5A): cluster 1 relating to the dryness of the skin (dry skin on the cheeks and on the forehead), cluster 2 relating to skin elasticity (elastosis, elasticity on the cheeks), wrinkles (upper-lip, crow's feet, underneath eyes, forehead, cheek folds), skin pigmentation and brightness (full face dyschromia, pigmentation of the malar area, full face brightness, papyraceus aspect on the cheeks), and cluster 3 relating to the oily nature of the skin (oily by touching on the cheeks and forehead, shiny on the cheeks and forehead, rough/scaly by touching on the cheeks and forehead, full face pore visibility, and erythrosis on the cheeks).

Univariate logistic models (FIG. 5B) showed increased risk of any of the (N=11) cluster 2 (wrinkles/elasticity-related) clinical signs with chronological age (OR ranging from 1.03 [1.01, 1.06] for full face brightness to 1.23 [1.18, 1.28] for elastosis on the full face), while a reduced risk of clinical signs was observed from cluster 3 (oily/shiny cluster) except for full face pore visibility, erythrosis on the cheeks, rough/scaly by touching on the cheeks and on the forehead (OR ranging from 0.95 [0.93, 0.98] for oily by touching on the cheeks to 0.94 [0.92, 0.96] for oily by touching on the forehead). The risk of clinical signs from cluster 1 (dryness) was not associated with chronological age.

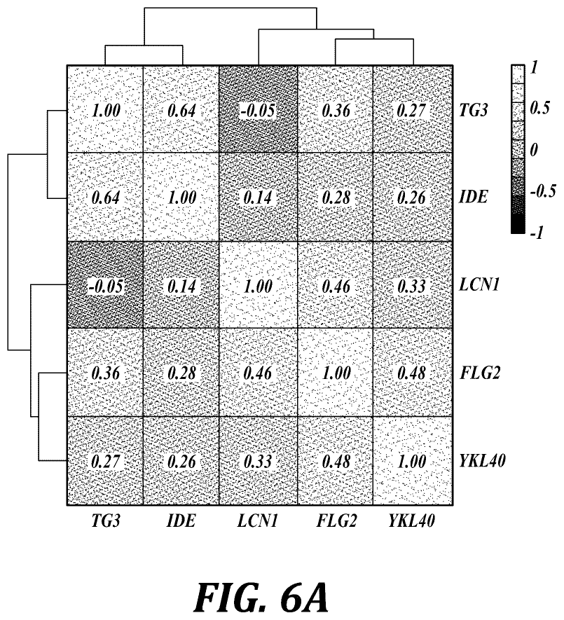

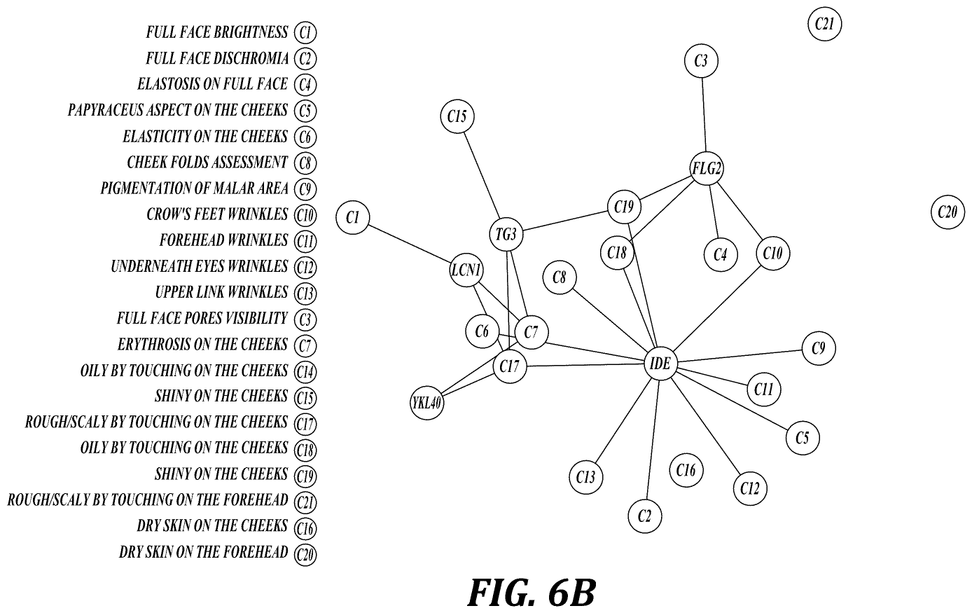

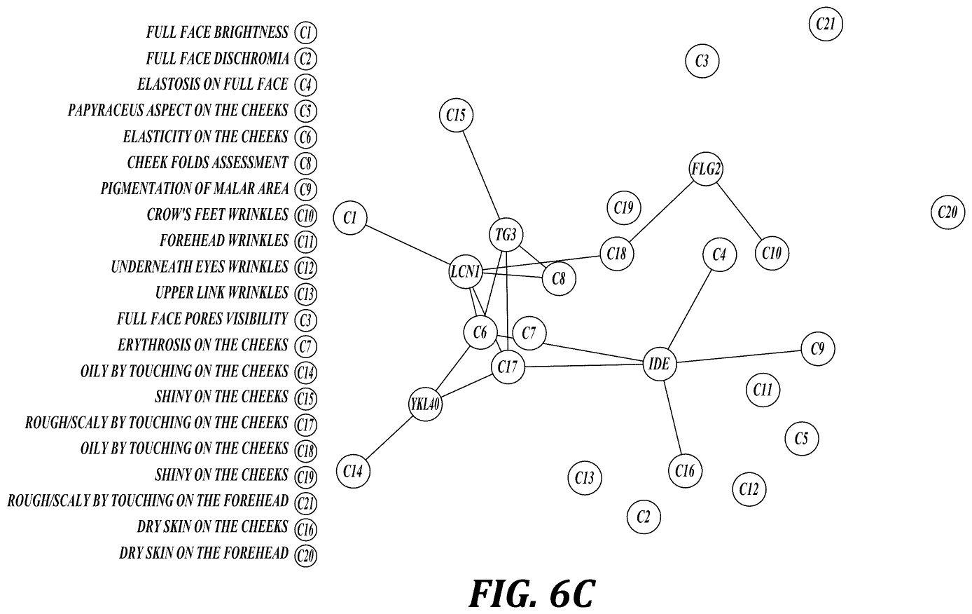

Proteins profiling. Skin levels of five proteins was measured in all study participants including Insulin Degrading Enzyme (IDE), Human Chitinase 3-like 1 (YKL40), Lipocalin-1 (LCN1), Transglutaminase-3 (TG3), and Filaggrin-2 (FLG2). A moderate-to-low pairwise correlation was observed across proteins ranging from −0.05 for LCN1-TG3 to 0.64 for the TG3-IDE (FIG. 6A). It was found that levels of LCN1 and FLG2 increased with chronological age (p<0.006) and that levels of TG3 (p<0.004) and IDE (p<0.00001) decreased with chronological age (FIG. 7). Univariate logistic regression models for the risk of each clinical signs as a function of the concentration of each protein separately suggested that higher concentration of FLG2 was associated with a lower risk of oily skin (by touching) on the forehead with OR of 0.59 [0.44, 0.79] and that higher concentration of IDE was associated with a lower risk of full face dyschromia with an OR of 0.59 [0.45, 0.77] and of crow's feet and underneath eyes wrinkle with ORs of 0.57 [0.43, 0.75] and 0.64 [0.48, 0.84], respectively. None of these associations survived adjustment for chronological age but chronological age-adjusted results suggested that (i) higher concentration of TG3 and IDE were associated with higher risk of dry skin on the cheeks and (ii) higher concentration of IDE were associated with lower risk of a shiny aspect of the cheeks.

To account for the correlation across proteins a series of (LASSO) penalized regression models was performed in a stability selection framework relating the measured concentrations of all proteins against each clinical sign separately (FIGS. 6B and 6C). It was found that conditionally on the skin concentration of all other proteins, IDE was associated with 8 wrinkle/elasticity-related clinical signs (cluster 2) and four clinical signs relating to the oily aspect of the skin (cluster 3, FIG. 6B). Concentrations of FLG2 were associated with three clinical signs relating to the oily aspect of the skin (cluster 3) and two wrinkles/elasticity-related outcomes (cluster 2), concentrations of LCN1 to two clinical signs relating to the oily aspect of the skin (cluster 3) and to one wrinkle/elasticity-related clinical sign (cluster 2). Concentrations of TG3 and YKL40 were exclusively associated with (N=4 and 2, respectively) clinical signs relating to the oily nature of the skin (cluster 3). Of the 21 clinical signs, those relating to the dryness of the skin (cluster 1) were not associated with any protein along with oily by touching on the cheeks and rough/scaly by touching on the forehead. Most clinical signs (N=12) were associated with a single protein, two (crow's feet wrinkle, and oily by touching on the forehead) with (the same) two proteins (FLG2 and IDE), one (shiny on the forehead) with three proteins and all proteins except FLG2 which was jointly associated with erythrosis on the cheeks and with rough/scaly by touching on the cheeks. Most of these associations, especially those involving IDE and those involving crow's feet and UE wrinkles-related signs, could be explained by chronological age and were not selected in the models adjusted for chronological age (FIG. 6C). Models adjusted for chronological age showed that (i) YKL40, LCN1, TG3 and IDE jointly and independently of chronological age contributed to the risk of rough/scaly by touching on the cheek and of elasticity on the cheeks, (ii) TG3 and LCN1 to the risk of cheeks folds, (iii) LCN1 and FGL2 to the risk of oily by touching on the forehead, and (v) either a single (N=8) or no protein (N=9) were contributing beyond chronological age to the risk of the other clinical signs. In the chronological age-adjusted model it was also found that the risk of dry skin on the cheeks was associated with concentrations of IDE.

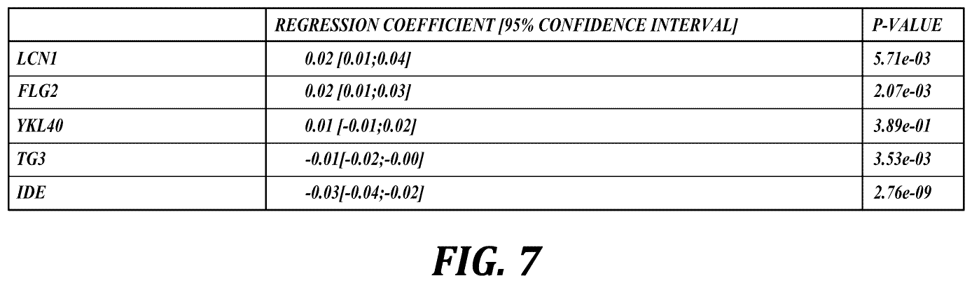

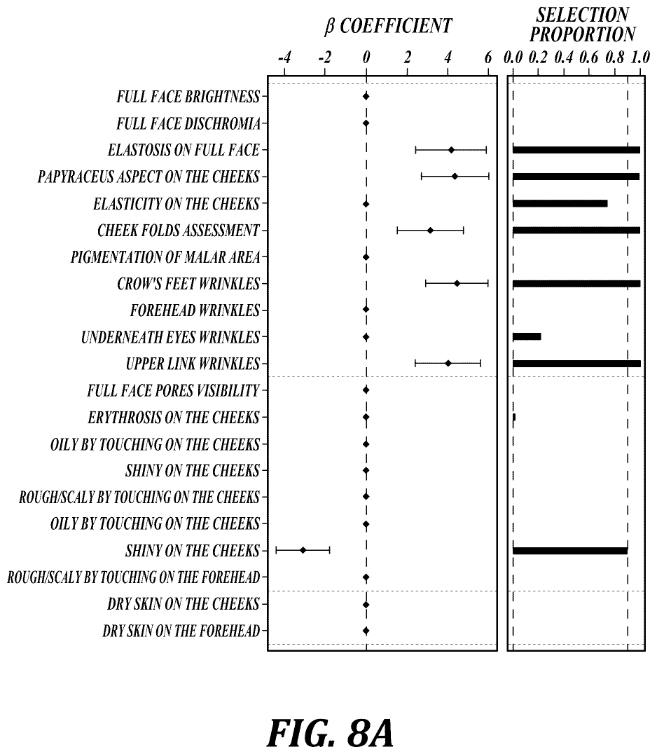

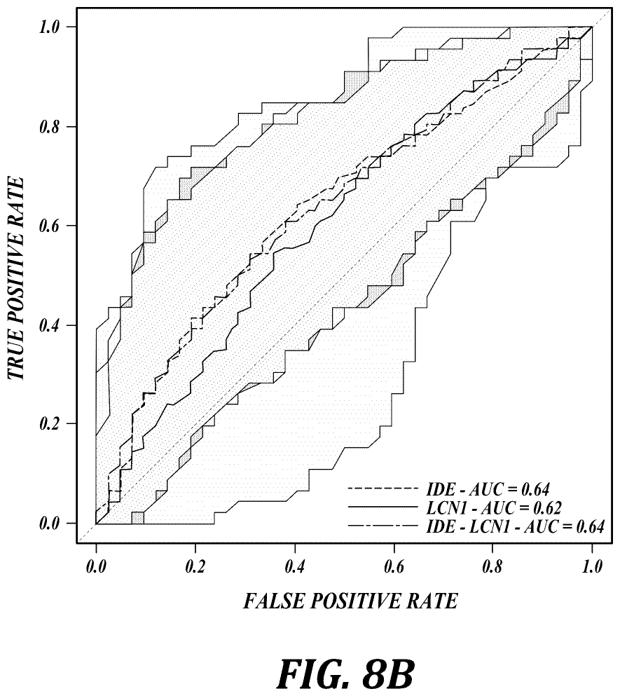

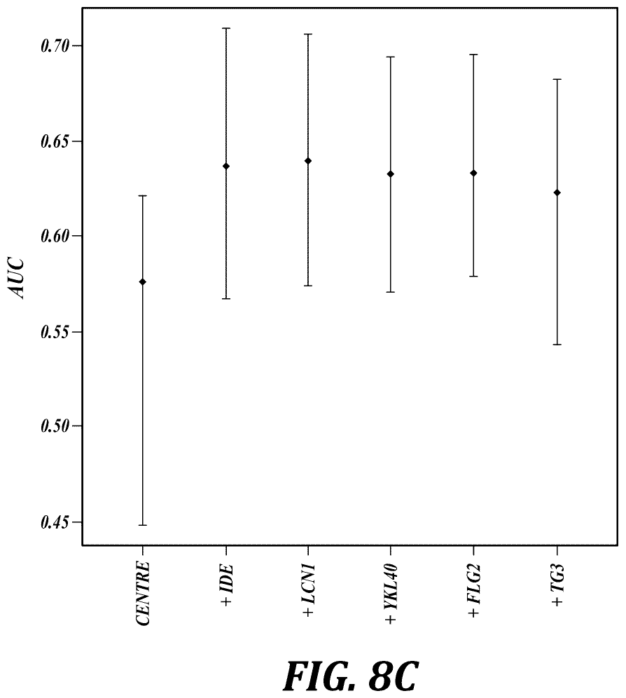

Clinical age and its determinants. Clinical age was defined as the part of chronological age that could be explained by the clinical profile of an individual. To account for the correlation across clinical signs a similar LASSO model calibrated via stability was adopted to identify the set of clinical signs jointly explanatory of chronological age. The stability selection LASSO approach identified that five wrinkle/elasticity-related clinical signs (cluster 2) were jointly and positively related to chronological age (including elastosis on the full face, papyraceus on the cheeks, cheek folds, crow's feet wrinkle and upper lip wrinkles) (FIG. 8A). Conditional on these clinical signs, it was also found that shiny on the forehead was negatively associated with chronological age. Altogether the 6 selected clinical signs explained 59% of the variance of chronological age.

Age A/D was defined as the difference between the chronological age and the clinical age. A negative value of this difference indicates an apparent age lower (age deceleration) than the chronological age and positive value indicates accelerated aging. Univariate analyses relating Age A/D and concentrations of each of the proteins separately showed that higher concentrations of LCN1 were associated with age acceleration, and higher concentration of TG3 (β=−1.40, p=8.7×10⁻⁴) and more markedly of IDE (β=−1.74, p=3.3×10⁻⁶) were associated with age deceleration (FIG. 9).

Stability selection models only identified concentrations of IDE as a predictor of Age A/D (selection proportion of 0.91). Selection proportion for LCN1 was 0.63 and did not reach the calibrated threshold of 0.89. When recoding Age A/D as a binary variable defined as true for a clinical age higher than the chronological age, both LCN1 and IDE were selected to be jointly predictive of Age A/D status with selection proportion greater than 0.9. ROC analyses based on logistic regression for the binary Age A/D indicator and recalibrated in 1000 independent validation sets each including 25% of the study population indicated that IDE was yielding an AUC of 0.64, LCN1 an AUC of 0.62, and that LCN1 was only modestly improving the model performance over that of IDE (FIG. 8B) and that the addition of any variable beyond these two was not further improving the model (FIG. 8C). Consistently, the models sequentially adding proteins in descending order of their selection proportion showed that IDE improved the AUC of the model from 0.611 for the model only including center to 0.637 and further to 0.641 while including LCN1. The addition of any other proteins did not improve the performance of the model, hence suggesting an efficient calibration of the model. Overall, these results suggest that LCN1, TG3, and IDE are three skin protein biomarkers associated with accelerated skin aging.

### Discussion

In this example, the levels of 5 targeted proteins from the stratum corneum were related to a series of 21 expert-scored clinical signs, including skin dryness, pore visibility, wrinkles, and oily nature of the skin. The descriptive analyses showed that the 21 scored clinical signs had strong co-occurrence patterns and clustering analyses identified 3 main clusters relating mainly to (i) the dryness of the skin, (ii) the skin elasticity and wrinkles, and (iii) the oily nature of the skin. Clinical signs from these different classes showed differential associations with chronological age with stronger associations unsurprisingly observed in clinical signs relating to the elasticity of the skin and presence of wrinkles. Clinical signs relating to the oily nature of the skin appeared to be overall inversely associated with chronological age, and dryness of the skin was not found associated with chronological age. Of the five proteins measured in the stratum corneum, the concentrations of LCN1 and FLG2 were found to increase with chronological age, while concentrations of TG3 and IDE were found to decrease with chronological age. Multivariate analyses regressing the five proteins against each clinical signs showed that IDE was associated with many clinical signs, in particular those relating to wrinkles/elasticity of the skin, and to the oily nature of the skin. Most of the former associations could be explained by chronological age, while most of the latter survived adjustment for chronological age. In the chronological age adjusted models, the results suggested that stratum corneum concentrations of IDE were, independently of chronological age, associated with five clinical signs, LCN1 and TG3 to three clinical signs, YKL40 to three clinical signs, and FLG2 to two clinical signs. Despite the strong co-occurrence patterns across clinical signs, there was limited overlap in the clinical signs associated with proteins and most clinical signs were explained by a single protein. Only shiny on the forehead and erythrosis on the cheeks were associated with three or more proteins, suggesting possibly more complex biological mechanisms at stake. In order to account for the co-occurrence of clinical signs clinical age was defined as the part of the chronological age that could be explained by the clinical signs. From this a measure of Age A/D was derived as the difference between the clinical and chronological ages. Stratum corneum concentrations of IDE (negatively) and LCN1 (positively) were found to be jointly associated to overall accelerated skin aging and can suggest potential determinants of skin aging or can point to specific pathways involved in the early onset of skin aging signs.

This example is believed to be the first to relate levels of LCN1 and aging, and the finding of IDE being associated with accelerated aging is in keeping with previous reporting of insulin clearance and accumulation of advanced glycation end products in the skin. For example, decreased concentration of IDE could lead to accelerated global aging concerns such as deregulated nutrient sensing in skin aging. This example represents the largest available data with detailed clinical signs assessment and stratum corneum proteins measurements, and the stability selection approaches used were able to detect biomarkers of facial skin features and aging. In addition, these approaches can be extended to (i) additional cohorts, (ii) participants of different skin types, and (iii) additional stratum corneum proteins. Furthermore, clinical skin age can be further evaluated with use of facial images. This example provides novel evidence linking the stratum corneum concentrations of IDE, LCN1, and TG3 as being related to skin clinical features and aging. These represent useful determinants of skin aging and potential targets for future interventions and treatments.

### Methods

In vivo clinical study design. A randomized multicentric clinical study was conducted on European volunteers, in France (Paris and Besançn) and in Romania. Each participant provided written informed consent prior to any procedure. A total of 376 healthy women, divided in 3 centers were included, aged 36 to 73 years old, skin phototype II or III according to Fitzpatrick's classification. The main inclusion criteria were: non-smoker or smoking less than 5 cigarettes per day, absence of suntan, absence of dermatological disorder affecting the face (i.e., vitiligo, acne, rosacea, or melasma), absence of cosmetic or surgical procedures on the face. Inclusion has been done to respect the same stratification according to 10 years age range in every center, i.e. 20 women between 36 and 45 years old (y.o.) 40 women between 46 and 55 y.o., 40 between 56 and 65 and 20 between 66 and 75 y.o. Participants reporting current or past use (for 1 week or more over the 8 weeks prior to invitation) of systemic or topical drugs or cosmetics such as antibiotics, anti-inflammatory drug, corticoids, retinoids, alpha hydroxyl-acids, vitamin C, benzoyl peroxide or any anti acne or antiseborrheic products were excluded. All eligible volunteers were provided a gentle cleansing product (Lipikar, La Roche Posay) to standardize their face cleansing on the evening prior to the evaluation visit. Volunteers did not wash, nor did they apply any product on their face in the morning prior to the visit.

In vivo clinical assessments. Dermatologists involved in the multicentric study were trained at the same time altogether by the same expert to be aligned on clinical assessments realized with an Evalux Bench® (Cosderma, Bordeaux, France) table to guarantee standardized conditions of lighting. Some evaluations, such as brightness, pores visibility, dischromia (skin tone heterogeneity) and elastosis, were performed using a 6 grades scale, on full face. Papyraceus aspect of the skin (texture), elasticity and erythrosis were assessed on the cheek with the 6 grades scale. Wrinkles (crow's feet, forehead, underneath eye, cheek folds) and pigmentation (malar area) assessments were done using referential Skin Aging Atlas. Skin type, including oiliness, shininess, dryness and roughness were assessed by touchy on cheek and forehead, using a 4 grades scale. For analysis, each clinical scoring of 21 clinical signs evaluated was dichotomized in 2 equal-sized groups “0” for absence and “1” for presence of the clinical sign.

D-squames sampling and protein concentration measurements. Stratum corneum layers samples were collected on the cheeks of these women using D-squame D100 (CuDerm Corporation, Dallas, Texas, USA). These were used to measure the stratum corneum levels of 5 prioritized proteins found associated with age in a previous study. These included filaggrin-2 (FLG2), a cytoplasmic protein involved in the cell adhesion process in the cornified cell layers; lipocalin-1 (LCN1) an extra-cellular protein playing an antimicrobial role; chitinase-3-like protein 1 (YKL40) a protein involved in the allergic skin inflammatory processes; protein-glutamine gamma-glutamyltransferase E (TGM3), a cytoplasmic protein involved in the formation of the cornified envelope of the skin; and Insulin Degrading Enzyme (IDE), a multifunctional enzyme implicated in the degradation of hormones (insulin) and other peptides such as amyloid β20. D-squames were incubated 2 min in 550 μL of 50 mM Tris, 150 mM NaCl, pH 8.0. This enabled the solubilization of the 5 example proteins of interest (LCN1, FLG2, YKL40, TG3, IDE), and subsequently their dosage on microfluidic cartridges using the FREND instrument (NanoEnTek) in a fast sandwich ELISA type assay.

Statistical analyses. A Principal Component Analysis (PCA) of the concentrations of 5 proteins measured in stratum corneum was performed and the data represented along the first 3 PC explaining 83.7% of the variance in the data to identify possibly outlying observations. Prevalence of each of the 21 dichotomized clinical signs was calculated in the full study population and separately for (i) four broad age groups: 36-45 years, 46-55 years, 56-65 years, and 66 years and over, and (ii) the three recruitment centers. Differences in prevalence were tested by age group or center using Chi-squared test and the corresponding p-value reported. To explore the co-occurrence of the 21 clinical signs hierarchical clustering with complete linkage using Jaccard's index was used as a measure of similarity. The number of clusters was determined as the one providing the largest decrease in the within-cluster Jaccard index. The risk of each of the clinical signs was investigated separately using a logistic model for clinical sign status using chronological age as predictor and adjusting for recruitment center. The concentrations of the five proteins measured in stratum corneum were normalized and centered to ensure comparability of the effect size estimates and were related (as outcome) to age (as predictor) using a linear model adjusted for recruitment center. The protein concentrations were then related to each (binary) clinical signs using two series of logistic regression models without and with adjustment for chronological age. To identify a sparse set of proteins that are jointly and complementarily predictive of each (N=21) clinical signs, a series of least absolute shrinkage and selection operator (LASSO) logistic models was used in a stability selection framework. Briefly, the model was run on 1000 independent 50% subsamples of the population, and for a given value of the penalty the per-protein selection proportion was derived across the 1000 models as measure of clinical signs importance. The two hyper-parameters of the model controlling the sparsity (the penalty term) and the stability (the selection proportion above which a protein is considered as stably selected) were calibrated jointly using a likelihood-based stability score. Results for the (N=21) stability selection logistic LASSO were plotted as a network where edges represent stably selected proteins jointly associated with each clinical sign. This model was subsequently adjusted (via non-penalization) for chronological age. Using a similar stability LASSO regression for the 21 clinical signs against chronological age, the dermatological age was defined as the part of clinical age that could be explained with a sparse set of clinical signs. Difference between the dermatological age and chronological age defined the skin Age A/D, which was subsequently related to the 5 proteins measures using univariate linear and stability selection LASSO models. A binary skin Age A/D indicator indicated if the participant had a higher apparent than chronological age was finally regressed against (i) each of the proteins separately using logistic regression and (ii) all the 5 proteins measured using a stability selection logistic LASSO to identify which proteins were jointly predictive of accelerated skin aging. Stability selection LASSO regressions were fitted in a training set of 50% of the participants. A series of logistic models with stably selected variables as predictors were fitted against binary Age A/D indicator in N=1000 recalibration sets with 25% of unseen participants. Receiver Operating Characteristics (ROC) curves were constructed in the (N=1000) remaining 25% of participants (test set) and reported pointwise median, 5th and 95th percentiles of the True and False Positive Rates and Area Under the Curve (AUC). Statistical analyses were performed using R version 4.2.221.

### Non-Limiting Embodiments

While general features of the disclosure are described and shown and particular features of the disclosure are set forth in the claims, the following non-limiting embodiments relate to features, and combinations of features, that are explicitly envisioned as being part of the disclosure. The following non-limiting Embodiments contain elements that are modular and can be combined with each other in any number, order, or combination to form a new non-limiting Embodiment, which can itself be further combined with other non-limiting Embodiments.

Embodiment 1. A system configured for management of skin health of an individual, the system comprising: circuitry configured to receive a measurement of one or more skin biomarkers of the individual and compute, based on the measurement, a predicted beauty outcome that is predicted to occur due to a course of action; circuitry configured to receive a desired beauty outcome for the individual and compute, based on the predicted beauty outcome and the desired beauty outcome, whether the course of action would advance the skin health of the individual toward the desired beauty outcome for a responsiveness metric; and circuitry configured to recommend or not recommend the course of action to the individual, based on the responsiveness metric, such that the skin health of the individual is advanced toward the desired beauty outcome.

Embodiment 2. The system of Embodiment 1 or any other Embodiment, wherein the measurement of the one or more skin biomarkers of the individual is transmitted from a measurement device and received by a computational device.

Embodiment 3. The system of any one of Embodiments 1-2 or any other Embodiment, wherein the measurement device comprises an on-site biomarker reader configured for on-site measurement of the one or more skin biomarkers of the individual.

Embodiment 4. The system of any one of Embodiments 1-3 or any other Embodiment, wherein the computational device comprises a networked server, a cloud computing system, a smartphone, a tablet, a desktop computer, a laptop computer, a smart watch, a wearable device, or any combination thereof.

Embodiment 5. The system of any one of Embodiments 1˜4 or any other Embodiment, wherein a first threshold of the measurement corresponds with a positive responsiveness metric and predicted responsiveness of skin of the individual to the course of action, and wherein a second threshold of the measurement corresponds with a negative responsiveness metric and predicted non-responsiveness of skin of the individual to the course of action.

Embodiment 6. The system of any one of Embodiments 1-5 or any other Embodiment, wherein the course of action comprises: a lifestyle change, a cessation of a negative habit, a reinforcement of a positive habit, a selection of a product, a selection of a service, an application of a product to skin of the individual, or any combination thereof.

Embodiment 7. The system of any one of Embodiments 1-6 or any other Embodiment, wherein the desired beauty outcome is computed as an assumed desired beauty outcome for the individual based on one or more characteristics of the individual, as an expressed desired beauty outcome based on one or more inputs from the individual, or any combination thereof.

Embodiment 8. The system of any one of Embodiments 1-7 or any other Embodiment, comprising circuitry configured to receive an imagery measurement of skin of the individual, an electromechanical measurement of skin of the individual, or both, and circuitry configured to compute the responsiveness metric based on the imagery measurement, the electromechanical measurement, or both.

Embodiment 9. The system of any one of Embodiments 1-8 or any other Embodiment, wherein the imagery measurement is transmitted from an image capture device and received by a computational device, and wherein the electromechanical measurement is transmitted from an electromechanical device and received by the computational device.

Embodiment 10. The system of any one of Embodiments 1-9 or any other Embodiment, comprising circuitry configured to receive a plurality of measurements of a plurality of skin biomarkers of the individual and compute, based on the plurality of measurements, the predicted beauty outcome that is predicted to occur due to the course of action.

Embodiment 11. The system of any one of Embodiments 1-10 or any other Embodiment, wherein the circuitry computes the predicted beauty outcome based on measurements of one or more relatively up-regulated skin biomarkers, measurements of one or more relatively down-regulated skin biomarkers, or any combination thereof.

Embodiment 12. The system of any one of Embodiments 1-11 or any other Embodiment, wherein the responsiveness metric is computed based at least in part on: a skin health characteristic of skin of the individual selected from the group consisting of: a skin texture, a cornification level, a presence or absence of an eye wrinkle, a level of insulin metabolism, a level of skin evenness or elastosis, a degree of loss of elasticity of skin, an antimicrobial property, a presence or absence of a dilated pore, a degree of inflammation, and any combination thereof; a predicted response of skin of the individual to a retinol treatment selected from the group consisting of: retinol (vitamin A1), C-beta-D-xylopyranoside-2-hydroxy-propane (C-Xyloside or ProXylane®), azelaic acid, and any combination thereof; a longevity characteristic of the individual selected from the group consisting of: a degree of inflammation, a level of stem cell exhaustion, a degree of DNA instability, a degree of proteostasis, a degree of epigenetic change, a level of microbiome change, a level of mitochondrial function, a level of cellular communication, a level of cellular senescence, and any combination thereof.

Embodiment 13. The system of any one of Embodiments 1-12 or any other Embodiment, wherein the responsiveness metric is computed based at least in part on a skin health characteristic of skin of the individual and the one or more skin biomarkers comprises a protein selected from the group consisting of: FLG2, IDE, TG3, LCN, YKL40, and any combination thereof.

Embodiment 14. The system of any one of Embodiments 1-13 or any other Embodiment, wherein the responsiveness metric is computed based at least in part on a predicted response of skin of the individual to a retinol treatment and the one or more skin biomarkers comprises a protein selected from the group consisting of: RARRES1, ALDH3A2, NQO2, and any combination thereof.

Embodiment 15. The system of any one of Embodiments 1-14 or any other Embodiment, wherein the responsiveness metric is computed based at least in part on a longevity characteristic of the individual and the one or more skin biomarkers comprises a protein selected from the group consisting of: LMNA, GLB1, SERPINA1, LMNB1, PI3, CAT, SOD1, CDH1, CD44, RHOA, CDC42, TAP1, FLG, KRT1, one or more KRTs, KRT14, KRT15, KRT16, KRT17, IL-1α, IL-18, KLK5, KLK7, and any combination thereof.

In the foregoing description, specific details are set forth to provide a thorough understanding of exemplary embodiments of the present disclosure. It will be apparent to one skilled in the art, however, that the embodiments disclosed herein may be practiced without embodying all of the specific details. In some instances, well-known process steps have not been described in detail in order not to unnecessarily obscure various aspects of the present disclosure. Further, it will be appreciated that embodiments of the present disclosure may employ any combination of features described herein.

The present application may reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 10% of the stated value. For the purposes of the present disclosure, the phrase “at least one of A and B” is equivalent to “A and/or B” or vice versa, namely “A” alone, “B” alone, or “A and B.” Similarly, the phrase “at least one of A, B, and C,” for example, means (A), (B), (C), (A and B), (A and C), (B and C), or (A, B, and C), including all further possible permutations when greater than three elements are listed.

It should be noted that for purposes of this disclosure, terminology such as “upper,” “lower,” “vertical,” “horizontal,” “fore,” “aft,” “inner,” “outer,” “front,” “rear,” etc., should be construed as descriptive and not limiting the scope of the claimed subject matter. Further, the use of “including,” “comprising,” or “having” and variations thereof herein is meant to encompass the items listed thereafter and equivalents thereof as well as additional items. Unless limited otherwise, the terms “connected,” “coupled,” and “mounted” and variations thereof herein are used broadly and encompass direct and indirect connections, couplings, and mountings.

Throughout this specification, terms of art may be used. These terms are to take on their ordinary meaning in the art from which they come, unless specifically defined herein or the context of their use would clearly suggest otherwise.

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

The embodiments of the invention in which an exclusive property or privilege is claimed are defined as follows:

1. A system configured for management of skin health of an individual, the system comprising:
circuitry configured to receive a measurement of one or more skin biomarkers of the individual and compute, based on the measurement, a predicted beauty outcome that is predicted to occur due to a course of action;
circuitry configured to receive a desired beauty outcome for the individual and compute, based on the predicted beauty outcome and the desired beauty outcome, whether the course of action would advance the skin health of the individual toward the desired beauty outcome for a responsiveness metric; and
circuitry configured to recommend or not recommend the course of action to the individual, based on the responsiveness metric, such that the skin health of the individual is advanced toward the desired beauty outcome.

2. The system of claim 1, wherein the measurement of the one or more skin biomarkers of the individual is transmitted from a measurement device and received by a computational device.

3. The system of claim 2, wherein the measurement device comprises an on-site biomarker reader configured for on-site measurement of the one or more skin biomarkers of the individual.

4. The system of claim 2, wherein the computational device comprises a networked server, a cloud computing system, a smartphone, a tablet, a desktop computer, a laptop computer, a smart watch, a wearable device, or any combination thereof.

5. The system of claim 1, wherein a first threshold of the measurement corresponds with a positive responsiveness metric and predicted responsiveness of skin of the individual to the course of action, and wherein a second threshold of the measurement corresponds with a negative responsiveness metric and predicted non-responsiveness of skin of the individual to the course of action.

6. The system of claim 1, wherein the course of action comprises: a lifestyle change, a cessation of a negative habit, a reinforcement of a positive habit, a selection of a product, a selection of a service, an application of a product to skin of the individual, or any combination thereof.

7. The system of claim 1, wherein the desired beauty outcome is computed as an assumed desired beauty outcome for the individual based on one or more characteristics of the individual, as an expressed desired beauty outcome based on one or more inputs from the individual, or any combination thereof.

8. The system of claim 1, comprising circuitry configured to receive an imagery measurement of skin of the individual, an electromechanical measurement of skin of the individual, or both, and circuitry configured to compute the responsiveness metric based on the imagery measurement, the electromechanical measurement, or both.

9. The system of claim 8, wherein the imagery measurement is transmitted from an image capture device and received by a computational device, and wherein the electromechanical measurement is transmitted from an electromechanical device and received by the computational device.

10. The system of claim 1, comprising circuitry configured to receive a plurality of measurements of a plurality of skin biomarkers of the individual and compute, based on the plurality of measurements, the predicted beauty outcome that is predicted to occur due to the course of action.

11. The system of claim 10, wherein the circuitry computes the predicted beauty outcome based on measurements of one or more relatively up-regulated skin biomarkers, measurements of one or more relatively down-regulated skin biomarkers, or any combination thereof.

12. The system of claim 1, wherein the responsiveness metric is computed based at least in part on:
a skin health characteristic of skin of the individual selected from the group consisting of: a skin texture, a cornification level, a presence or absence of an eye wrinkle, a level of insulin metabolism, a level of skin evenness or elastosis, a degree of loss of elasticity of skin, an antimicrobial property, a presence or absence of a dilated pore, a degree of inflammation, and any combination thereof;
a predicted response of skin of the individual to a retinol treatment selected from the group consisting of: retinol (vitamin A1), C-beta-D-xylopyranoside-2-hydroxy-propane (C-Xyloside or ProXylane®), azelaic acid, and any combination thereof;
a longevity characteristic of the individual selected from the group consisting of: a degree of inflammation, a level of stem cell exhaustion, a degree of DNA instability, a degree of proteostasis, a degree of epigenetic change, a level of microbiome change, a level of mitochondrial function, a level of cellular communication, a level of cellular senescence, and any combination thereof.

13. The system of claim 1, wherein the responsiveness metric is computed based at least in part on a skin health characteristic of skin of the individual and the one or more skin biomarkers comprises a protein selected from the group consisting of: filaggrin 2 (FLG2), Insulin-degrading enzyme (IDE), epidermal transglutaminase (TG3), lipocalin (LCN), Chitinase-3-like protein 1 (YKL40), and any combination thereof.

14. The system of claim 1, wherein the responsiveness metric is computed based at least in part on a predicted response of skin of the individual to a retinol treatment and the one or more skin biomarkers comprises a protein selected from the group consisting of: Retinoic acid receptor responder protein 1 (RARRES1), Aldehyde dehydrogenase family 3 member A2 (ALDH3A2), N-ribosyldihydronicotinamide:quinone dehydrogenase 2 (NQO2), and any combination thereof.

15. The system of claim 1, wherein the responsiveness metric is computed based at least in part on a longevity characteristic of the individual and the one or more skin biomarkers comprises a protein selected from the group consisting of: lamin A/C (LMNA), galactosidase beta 1 (GLB1), serpin family A member 1 (SERPINA1), lamin B1 (LMNB1), peptidase inhibitor 3 (PI3), catalase (CAT), superoxide dismutase 1 (SOD1), cadherin 1 (CDH1), CD44 molecule (IN blood group) (CD44), ras homolog family member A (RHOA), cell division cycle 42 (CDC42), transporter 1, ATP binding cassette subfamily B member (TAP1), filaggrin (FLG), keratin 1 (KRT1), one or more keratin genes (KRTs), keratin 14 (KRT14), keratin 15 (KRT15), keratin 16 (KRT16), keratin 17 (KRT17), interleukin-1 alpha (IL-1α), interleukin-18 (IL-18), Kallikrein Related Peptidase 5 (KLK5), Kallikrein Related Peptidase 7 (KLK7), and any combination thereof.
